(19) United States
(12) Patent Application Publication
(10) Pub. No.: US 2010/0260704 A1
(43) Pub. Date: Oct. 14, 2010

(54) HUMAN INTERFERON-GAMMA (INFGAMMA) VARIANTS

(75) Inventors: Jose Berenguer, Madrid (ES); Marc Delcourt, Paris (FR); Héléne Chautard, Paris (FR); Thierry Menguy, Strasbourg (FR)

Correspondence Address: SALIWANCHIK LLOYD & SALIWANCHIK;A PROFESSIONAL ASSOCIATION, PO Box 142950, GAINESVILLE, FL, 32614, US

(73) Assignee: BIOMETHODES, Evry (FR)

(21) Appl. No.: 12/281,192

(22) PCT Filed: Mar. 8, 2007
(86) PCT No.: PCT/FR2007/000419

§ 371 Date: Aug. 29, 2008

(30) Foreign Application Priority Data
Mar. 8, 2006 (FR) 0602064
Apr. 10, 2006 (FR) 0603150

(51) Int. Cl. A61K 38/20; A61K 38/21; C12P 21/06; C12N 5/10; C12N 15/63; C07K 14/57; C07H 21/04; A61P 37/00; A61P 11/06; A61P 11/00; A61P 31/04; A61P 35/04; A61P 19/10; A61P 19/02

(52) U.S. Cl. 424/85.2; 424/85.5; 435/69.51; 435/325; 435/320.1; 530/351; 536/23.52

(57) ABSTRACT

The present invention relates to human interferon gamma variants with improved thermostability, to a nucleic acid encoding said variants, to a pharmaceutical composition containing them, and to their use for the treatment of a viral infection and of cancer.

### INTRODUCTION

The present invention relates to the field of protein improvement. The invention relates to the improvement of human interferon γ (IFNγ), and to compositions comprising an improved IFNγ, to a nucleic acid encoding same, and to the uses thereof.

IFNγ is a cytokine composed of 166 amino acids. The molecule contains a signal peptide enabling membrane translocation and secretion, a cleavage site and a portion corresponding to the so-called mature protein. The signal peptide of IFNγ is variously reported by different authors to be composed of the first 23 or the first 20 amino acid residues. In fact, there is a doubt in the literature as to the presence of the amino acid triplet Cys-Tyr-Cys (CYC) at the N-terminal end of the mature sequence. IFNγ exists as a homodimer of two noncovalently bound subunits. Each subunit has two N-glycosylation sites (positions 48 and 120 of the 166 amino acid precursor). If the CYC residues are not taken into account, there are no disulfide bridges or cysteines in the mature protein in vivo. Each monomer has six alpha helices, with a compact region composed of the first four alpha helices closest to the N-terminal (alpha helices A, B, C and D), and a C-terminal region composed of two isolated alpha helices and in close interaction with the second IFNγ monomer.

IFNγ is the archetype of a pleiotropic cytokine with a broad spectrum of activities. Indeed, the interferons (IFNs) are endowed with activities such as inhibition of viral replication, inhibition of cell multiplication and induction of apoptosis.

In particular, macrophage stimulation by IFNγ induces the following responses:
increase in phagocytosis and bacterial killing (direct antimicrobial and antitumoral mechanisms);
stimulation of antigen-presenting and degradation pathways, expression of major histocompatibility complex (MHC) type I and II at the macrophage surface;
differentiation of B lymphocytes to antibody-secreting plasmocytes, which results in IgG production and complement activation;
activation of NO synthase giving rise to production of cytotoxic free oxygen radicals and NO; and/or
increase in the production of cytokines and endogeneous IFN.

IFNγ acts on T lymphocytes by promoting their differentiation thereby modulating the specific immune response.

By virtue of its broad spectrum of activity (antiviral, antiproliferative and immunomodulating), IFNγ has been developed as a therapeutic agent for the treatment of a wide variety of human diseases. Commercially available IFNγ (Actimmune and Biogamma) are currently used in two main therapeutic indications: chronic granulomatous disease and idiopathic pulmonary fibrosis, in combination with oral prednisolone. Many new, secondary therapeutic indications are currently in different clinical phases of development, in particular for their immunosuppressive role for example as an adjunct to pegylated IFNα/ribavirin in the treatment of hepatitis C. The following may also be mentioned: atypical mycobacterial infections; kidney cancer; osteopetrosis; systemic scleroderma; chronic hepatitis B; chronic hepatitis C; different viral infections including those due to papillomaviruses; septic shock; allergic dermatitis; rheumatoid arthritis; ovarian cancer; hepatic fibrosis; asthma; and lymphoma.

The main side effects of IFNs are dose-related and are therefore closely linked to the dosing schedule. Said effects are cumulative and worsen over time. In addition to acute toxicity which can cause malaise, nausea and vomiting shortly after injection (2-8 hours after a subcutaneous injection), the most common side effects are flu-like symptoms (chills, headache, fatigue), inflammatory reactions at the injection site and elevation of liver transaminases. The most serious adverse effects include depression, lymphocytopenia and rare instances of necrosis at the subcutaneous injection site. Patients receiving high doses of IFN may develop diabetes after the initiation of therapy. Furthermore, the tolerability of IFN injections is sometimes limited over time and is manifested by the development of neutralizing antibodies (in approximately 10-20% of patients).

The in vivo half-life of recombinant human IFNγ is only 25-35 minutes. For this reason efficient treatment with IFNγ involves frequent injections. When recombinant human IFNγ marketed under the name Actimmune (Intermune Inc.) came into use back in 1990, it became clear that there was a need to improve the half-life of IFNγ, all the more so since of all the interferons, IFNγ has the lowest thermostability. Indeed, a more stable IFNγ would allow a longer interval between injections, thus improving patient comfort. A more stable IFNγ would also enable better in vivo efficacy, since the in vivo effect results from a combination of the specific activity of the protein and its duration of action. The economic advantages of improving the stability of IFNγ are therefore obvious: a more stable molecule (either because it is a variant, or because it contains additives, or because the formulation has been improved, etc.) would make it possible to produce second generation medicaments that could replace the currently available agents. It might even be thought that a more stable IFNγ would expand the indications of the molecule: in fact, IFNα and β, among other molecules, are the reference treatments in certain pathologies, but IFNγ is not counted among them because its half-life is too short. Finally, in the longer term, it might be hoped that a more stable IFNγ will allow it to be formulated for different routes of administration (in particular an oral form).

IFNγ is already the subject of extensive research:

### Natural IFNγ Variants

Several natural mutants have been described. One such form is that which contains the N-terminal CYC sequence. Two natural variants of human IFNγ containing a point mutation have also been described: K29Q and R160Q (Nishi et al., J. Biochem. 97:153-159, 1985). However, these polymorphisms have so far not been associated with any effect.

### Artificial IFNγ Mutants

U.S. Pat. No. 4,832,959 discloses polypeptides with partial sequences of human IFNγ comprising residues 1-127, 5-146 and 5-127 of mature IFNγ and having the three additional amino acid residues CYC.

U.S. Pat. No. 6,120,762 discloses a peptide fragment comprising residues 118-157 of the IFNγ precursor and its use.

WO2004005341 provides methods for generating and producing a series of active IFNγ mutants comprising the 143 amino acids of the mature form of IFNγ without the CYC residues, with one variation comprising at least one of the mutations in the group S155 and S165 and at least one mutation in the group R160, R162 and R163. Said mutations would be useful in particular in the treatment of idiopathic pulmonary fibrosis.

Many variants truncated at different positions in the C-terminal region of IFNγ have been described. EP 0 219 781 discloses the use of partial human IFNγ sequences comprising amino acids 3-124 of the mature protein. The importance of the last 20 amino acid residues on the activity and stability of IFNγ has been and continues to be a subject of controversy. C-terminally truncated human IFNγ variants have been described by Slodowski et al., who produced truncated forms of different sizes (from 10 to 20 truncated amino acid residues) (Eur. J. Biochem. 202:1133-1140, 1991).

In EP 0 306870, IFNγ variants with significantly higher activity were generated by deleting from 7 to 11 C-terminal residues. Moreover, it is known that IFNγ production by mammalian cells results in a heterogeneous population of IFNγ polypeptides due to natural truncation by endo- and exo-proteases secreted by the producer host cell. One of the ways to resolve this production problem is disclosed in U.S. Pat. No. 6,958,388 and consists in producing a truncated IFNγ containing the first 155 amino acids of the whole protein associated for example with mutations improving the glycosylation of the molecule (S122T, E61N+S63T).

WO 2004/022593 analyses in silko the sequences of many therapeutic proteins, including IFNγ, for the existence of proteolytic sites susceptible to the proteases present in human serum (such as trypsin, Asp-N endoprotease, chymotrypsin and proline endopeptidase). The mutations which supposedly protect against the aforementioned proteases are the following: L53V, L53I, K57Q, K57N, K60Q, K60N, E61Q, E61N, E61H, E62Q, E62N, E62H, K78Q, K78N, K81Q, K81N, K84Q, K84N, D85Q, D85N, D86Q.

The improvements (in activity or stability) which said mutations may confer have not yet been experimentally verified for any of these mutants, so this remains at the level of scientific conjecture.

IFNγ Mutants with Improved Thermostability

IFNγ (in particular the monomeric form) is known to have low stability. This makes the wild-type protein highly sensitive to two parameters: acidic pH and temperature. It is expected that the increased stability of mutants in these non-physiological conditions will be associated with a similar increase in stability in physiological conditions. Studies have therefore focused on the search for mutants with increased thermostability, this parameter being the easiest to measure.

WO 92/08737 discloses IFNγ variants comprising an added methionine at position −1 of the N-terminal end, the first 132 amino acids of the mature sequence without the CYC residues, the 133rd amino acid being a leucine instead of a glutamine. Said variant, with truncation of the ten C-terminal amino acids of IFNγ, is named Delta 10 L or C10L. It purportedly has improved biological activity and slightly improved thermostability (mt 55° C.) as compared to wild-type IFNγ (mt 52-53° C.).

U.S. Pat. No. 4,898,931 discloses a series of IFNγ mutants produced in E. coli with truncation of the last nine C-terminal residues coupled with mutations of certain N- and C-terminal amino acids. Said mutations introduce disulfide bridges which then confer thermostable properties to these molecules all while preserving antiviral and antitumoral activity. In this patent, the CYC residues are part of the mature protein and variants of the cysteines in this triplet were produced.

| | |
|---|---|
| Wild-type (complete sequence) | 25% residual activity after 1 h at 50° C. |
| Mutation M157C + Delta 9 | 81% residual activity after 1 h at 50° C. |
| Mutation C21S-M157C + | 86% residual activity after 1 h at 50° C. |
| Delta 9 | |
| Mutation C23S-M157C + | 98% residual activity after 1 h at 50° C. |
| Delta 9 | |
| Mutation C21S-C23S- | 23% residual activity after 1 h at 50° C. |
| M157C + Delta 9 | |

U.S. Pat. No. 6,046,034 discloses thermostable human IFNγ variants in which cysteine pairs are incorporated at specific places in the IFNγ structure so as to create inter-monomeric and intra-monomeric disulfide bridges and thereby stabilize the IFNγ homodimer. The only cysteine pair which conserves the biological activity of IFNγ is E30C-S92C which links helices A and D of a same monomer, the other inter-monomeric cysteine pairs abolishing the biological activity of IFNγ. The mutants disclosed in this patent also have a truncated C-terminal corresponding to the Delta 10 mutant.

Modification of IFNγ by the addition of polymers has been reported by Kita et al. (Drug Des. Deliv. 6:157-167, 1990), and in EP 236987 and U.S. Pat. No. 5,109,120.

WO 99/03887 discloses variants of proteins belonging to the growth hormone structural superfamily (which includes IFNγ), wherein certain non-essential amino acid residues of the peptide structure have been replaced by a cysteine residue. IFNγ is mentioned as one example of a member of said superfamily but experimental modification of IFNγ is not disclosed.

WO 01/36001 discloses novel IFNγ molecules that have been modified by insertion of glycosylation sites and/or derivatized by moieties of the PEG type. Said molecules have improved properties such as an improved half-life and/or an improved bioavailability.

WO03002152 describes a pharmaceutical composition containing a sulfoalkyl ether cyclodextrin derivative of interferon which has improved stability.

Currently, none of said variants is available in the form of a medicament. This is why there is still a strong demand for an improved IFNγ, and in particular an IFNγ with higher stability in physiological conditions. Said increased stability in physiological conditions can be evaluated by an increase in stability at high temperature.

### SUMMARY OF THE INVENTION

The invention relates to a thermostable variant of human IFNγ or a functional fragment thereof.

In particular, the invention relates to a pharmaceutical composition comprising a thermostable variant of human IFNγ or a functional fragment thereof comprising at least one substutition selected in the group consisting of S63C, E62C, F159C, D99Y, E116C, L158C, S74G, R162C, S122D, L126P, N58R, and T95V, the variant not containing a non-peptide moiety attached to the residue(s) introduced by the first substutition(s). In one embodiment, the variant differs from a polypeptide having a sequence selected in the group consisting of sequences SEQ ID Nos. 2, 4 and 6 by at least 1, 2, 3, 4, 5, 6, 7, 8, 9 or 10 residues, preferably by 1, 2, 3, 4, 5, 6, 7, 8, 9 or 10 residues. In a particular embodiment, the variant has a single substitution. In another embodiment, the variant additionally comprises at least one other substitution selected in the group consisting of M157C, G41S and M100N. In particular, the variant may comprise or have a combination of two substitutions selected in the group consisting of S63C, E62C, F159C, D99Y, E116C, L158C, S74G, R162C, S122D, M100N, L126P, N58R, T95V, M157C and G41S. In a preferred embodiment, the variant comprises or has a combination of substitutions selected in the group consisting of S63C+E62C, S63C+F159C, S63C+D99Y, S63C+E116C, S63C+L158C, S63C+S74G, S63C+R162C, S63C+S122D, S63C+M100N, S63C+L126P, S63C+N58R, S63C+T95V, S63C+M157C, S63C+G41S, E62C+F159C, E62C+D99Y, E62C+E116C, E62C+L158C, E62C+S74G, E62C+R162C, E62C+S122D, E62C+M100N, E62C+L126P, E62C+N58R, E62C+T95V, E62C+M157C, E62C+G41S, F159C+D99Y, F159C+E116C, F159C+L158C, F159C+S74G, F159C+R162C, F159C+S122D, F159C+M100N, F159C+L126P, F159C+N58R, F159C+T95V, F159C+M157C, F159C+G41S, D99Y+E116C, D99Y+L158C, D99Y+S74G, D99Y+R162C, D99Y+S122D, D99Y+M100N, D99Y+L126P, D99Y+N58R, D99Y+T95V, D99Y+M157C, D99Y+G41S, E116C+L158C, E116C+S74G, E116C+R162C, E116C+S122D, E116C+M100N, E116C+L126P, E116C+N58R, E116C+T95V, E116C+M157C, E116C+G41S, L158C+S74G, L158C+R162C, L158C+S122D, L158C+M100N, L158C+L126P, L158C+N58R, L158C+T95V, L158C+M157C, L158C+G41S, S74G+R162C, S74G+S122D, S74G+M100N, S74G+L126P, S74G+N58R, S74G+T95V, S74G+M157C, S74G+G41S, R162C+S122D, R162C+M100N, R162C+L126P, R162C+N58R, R162C+T95V, R162C+M157C, R162C+G41S, S122D+L126P, S122D+N58R, S122D+T95V, S122D+M157C, S122D+M100N, S122D+G41S, L126P+N58R, L126P+T95V, L126P+M157C, L126P+M100N, L126P+G41S, N58R+T95V, N58R+M157C, N58R+M100N, N58R+G41S, T95V+M157C, T95V+M100N, T95V+G41S, M157C+M100N and M157C+G41S. In a more preferred embodiment, the variant comprises or has a combination of substitutions selected in the group consisting of S63C+E62C, S63C+F159C, S63C+D99Y, S63C+E116C, S63C+L158C, S63C+S74G, S63C+R162C, S63C+S122D, S63C+M100N, S63C+L126P, S63C+N58R, S63C+T95V, S63C+M157C. and S63C+G41S. In a more particularly preferred embodiment, the variant comprises or has the combination S63C+G41S. In a particular embodiment, the variant does not have a deletion of 1 to 11 C-terminal residues. In another particular embodiment, the variant has a deletion of 1 to 11 C-terminal residues. In a particular embodiment, the variant does not contain a non-peptide moiety selected in the group consisting of a polymer molecule, a lipophilic molecule and an organic derivatizing agent. In another particular embodiment, the variant does contain a non-peptide moiety selected in the group consisting of a polymer molecule, a lipophilic molecule and an organic derivatizing agent. The non-peptide moiety in question is more particularly a polymer molecule, preferably a polyethylene glycol. In a particular embodiment, the variant is glycosylated. In another particular embodiment, the variant is not glycosylated. In a particular embodiment, the pharmaceutical composition additionally comprises at least one other active agent. The at least one other active agent is preferably selected in the group consisting of an antibody, an antitumoral or chemotherapeutic agent, a glucocorticoid, an antihistamine, an adrenocortical hormone, an antiallergic agent, a vaccine, a bronchodilator, a steroid, a beta-adrenergic agent, an immunomodulating agent, a cytokine such as interferon alpha or beta, interleukin 1 or 2, TNF (tumor necrosis factor), hydroxyurea, an alkylating agent, a folic acid antagonist, a nucleic acid antimetabolite, a spindle poison, an antibiotic, a nucleotide analogue, a retinoid, a lipoxygenase and cyclooxygenase inhibitor, fumaric acid and its salts, an analgesic, a spasmolytic, and a calcium antagonist. In a preferred embodiment, the at least one other active agent is a type I interferon, in particular an interferon alpha or beta. The pharmaceutical composition may be formulated for administration by the oral, parenteral (for example subcutaneous, intramuscular, intravenous or intradermal), sublingual, topical, local, intratracheal, intranasal, transdennal, rectal, intraocular or intraauricular route.

The present invention also relates to a pharmaceutical composition according to the invention as medicament.

The present invention relates to a product comprising a pharmaceutical composition according to the invention and another active agent for a combined preparation for simultaneous, sequential or separate use as an antiviral, antiproliferative or immunomodulating medicament. Preferably, the other active agent is selected in the group consisting of an antibody, an antitumoral or chemotherapeutic agent, a glucocorticoid, an antihistamine, an adrenocortical hormone, an antiallergic agent, a vaccine, a bronchodilator, a steroid, a beta-adrenergic agent, an immunomodulating agent, a cytokine such as interferon alpha or beta, interleukin 1 or 2, TNF (tumor necrosis factor), hydroxyurea, an alkylating agent, a folic acid antagonist, a nucleic acid antimetabolite, a spindle poison, an antibiotic, a nucleotide analogue, a retinoid, a lipoxygenase and cyclooxygenase inhibitor, fumaric acid and its salts, an analgesic, a spasmolytic, and a calcium antagonist. In a preferred embodiment, the other active agent is a type I interferon, in particular an interferon alpha or beta. The two active agents may be administered by the same route of administration or by two different routes of administration. In a particular embodiment, the medicament is designed for treating a pathology selected in the group consisting of asthma, chronic familial granulomatous disease, idiopathic pulmonary fibrosis, an atypical mycobacterial infection, kidney cancer, osteopetrosis, systemic scleroderma, chronic hepatitis B or C, septic shock, allergic dermatitis, and rheumatoid arthritis.

The present invention also relates to the use of a pharmaceutical composition according to the invention for preparing an antiviral, antiproliferative or immunomodulating medicament. In a preferred embodiment, the medicament is for the treatment of a pathology selected in the group consisting of asthma, chronic familial granulomatous disease, idiopathic pulmonary fibrosis, an atypical mycobacterial infection, kidney cancer, osteopetrosis, systemic scleroderma, chronic hepatitis B or C, septic shock, allergic dermatitis, and rheumatoid arthritis.

The present invention relates to a nucleic acid encoding a thermostable IFNγ variant such as described in the aforementioned compositions. The invention also relates to an expression cassette of a nucleic acid according to the invention, a vector comprising a nucleic acid or an expression cassette according to the invention, and a host cell comprising a nucleic acid, an expression cassette or a vector according to the invention. The invention further relates to the use of one such nucleic acid, one such expression cassette, one such vector or one such host cell for producing a thermostable IFNγ variant such as described in the aforementioned compositions.

### BRIEF DESCRIPTION OF FIGURES AND TABLES

FIG. 1: pNCK vector used to generate mutant libraries and their selection in Thermus thermophilus.

FIG. 2: Functional analysis data for IFNγ single mutants selected in Thermus thermophilus: Thermostability analysis (% residual activity, panel A), relative total activity compared to wild-type protein (panel B) and improvement index per product (residual activity by relative total activity compared to wild-type/100, panel C).

FIG. 3: Functional analysis data for IFNγ single mutants derived from double and multiple positions selected in Thermus thermophilus: Thermostability analysis (% residual activity, panel A), relative total activity compared to wild-type protein (panel B) and improvement index per product (residual activity by relative total activity compared to wild-type/100, panel C)

FIG. 4: First part of functional analysis data for systematically generated IFNγ single point mutants with improved stability and/or activity—Thermostability analysis (% residual activity, panel A), relative total activity compared to wild-type protein (panel B) and improvement index per product (residual activity by relative total activity compared to wild-type/100, panel C).

FIG. 5: Second part of functional analysis data for systematically generated IFNγ single point mutants with improved stability and/or activity—Thermostability analysis (% residual activity, panel. A), relative total activity compared to wild-type protein (panel B) and improvement index per product (residual activity by relative total activity compared to wild-type/100, panel C).

FIG. 6: Third part of functional analysis data for systematically generated IFNγ single point mutants with improved stability and/or activity—Thermostability analysis (% residual activity, panel A), relative total activity compared to wild-type protein (panel B) and improvement index per product (residual activity by relative total activity compared to wild-type/100, panel C).

FIG. 7: Thermostability evaluation of human IFNγ protein variants by measuring in vitro half-life in a thermal denaturation kinetic study at 59° C. in the presence of an adjusted FCS concentration. Measurements consisted in following the amount of IFNγ activity conserved as a function of denaturation time at 59° C. These data were used to calculate the “in vitro half-life” of these molecules in these conditions. Half-life calculations are presented in Table 3.

FIG. 8: Pharmacokinetic profile of thermostable human IFNγ variants following intravenous administration. IFNγ levels were monitored by ELISA on serum collected from C57BL/6 mice after intravenous injection of 100 μl of a 10 μg/ml solution.

FIG. 9: Example of pharmacokinetic profile of thermostable human IFNγ variants following subcutaneous administration. IFNγ plasma concentrations were quantified by ELISA on serum collected from C57BL/6 mice after subcutaneous injection of 100 μl of a 6.7 μg/ml solution.

Table 1: Mutants identified in the primary selection of the INFγ library in Thermus thermophilus. Numbers correspond to the position of the mutation in the 166-residue precursor.

Table 2: Mutants validated by the secondary selection test in Thermus thermophilus. Numbers correspond to the position of the mutation in the 166-residue precursor.

Table 3: Summary of “in vitro” half-life calculations for thermostable IFNγ variants in the thermal denaturation studies at 59° C. and calculation of the ratio of improvement of the variant half-lives as compared to the half-life of wild-type IFNγ produced in CHO.

Table 4: Summary of terminal elimination half-life calculations for thermostable IFNγ variants during intravenous injection studies and calculation of the ratio of improvement of the variant half-lives as compared to the half-life of wild-type IFNγ produced in CHO. Terminal elimination half-lives (T1/2 i.v.) were calculated with the aid of Kinetica Vs 4.4 software using a non-compartmental IV bolus model.

Table 5: Summary of calculations of total area under the curve (AUC tot. s.c.) and elimination half-life (T1/2 s.c.) during subcutaneous injection studies. Calculation of the ratios of improvement of total AUC and half-life of the variants as compared to the total AUC and half-life of wild-type IFNγ produced in CHO. These parameters were calculated with the aid of Kinetica Vs 4.4 software.

### DETAILED DESCRIPTION OF THE INVENTION

The present invention relates to variants of human interferon gamma (IFNγ) whose stability, in particular thremostability, is increased as compared to wild-type IFNγ. The IFNγ protein variants of the invention were obtained by coupling the generation of a wide diversity of mutations produced by directed evolution, with a method for directly selecting variants displaying improved thermostability. The stability of the improved candidates to thermal denaturation as well as the conservation of their activity were validated by biological tests. The variants according to the invention represent alternatives to the recombinant INFγ currently in therapeutic use, particularly in the treatment of chronic granulomatous disease and idiopathic pulmonary fibrosis.

In light of the lingering doubt as to the presence of the CYC amino acid residues at the N-terminal end of the mature IFNγ protein, and the resultant doubt as to the numbering of amino acids in the mature protein, the numbering adopted in the present application will be that which takes into account all IFNγ residues including the signal peptide sequence (total amino acids numbered from 1 for the included N-terminal methionine signal peptide to 166 for the C-terminal glutamine of interferon—sequence SEQ ID No. 2). The position of the substitution in the other two forms (mature form without CYC, SEQ ID No. 4, or with CYC, SEQ ID No. 6) can be easily determined by those skilled in the art.

The following terminology is used to denote a substitution: C21G indicates substitution of the cysteine residue at position 21 of SEQ ID No. 2 by a glycine residue. The terms “substitution” and “mutation” are interchangeable. The sign “+” indicates a combination of two substitutions.

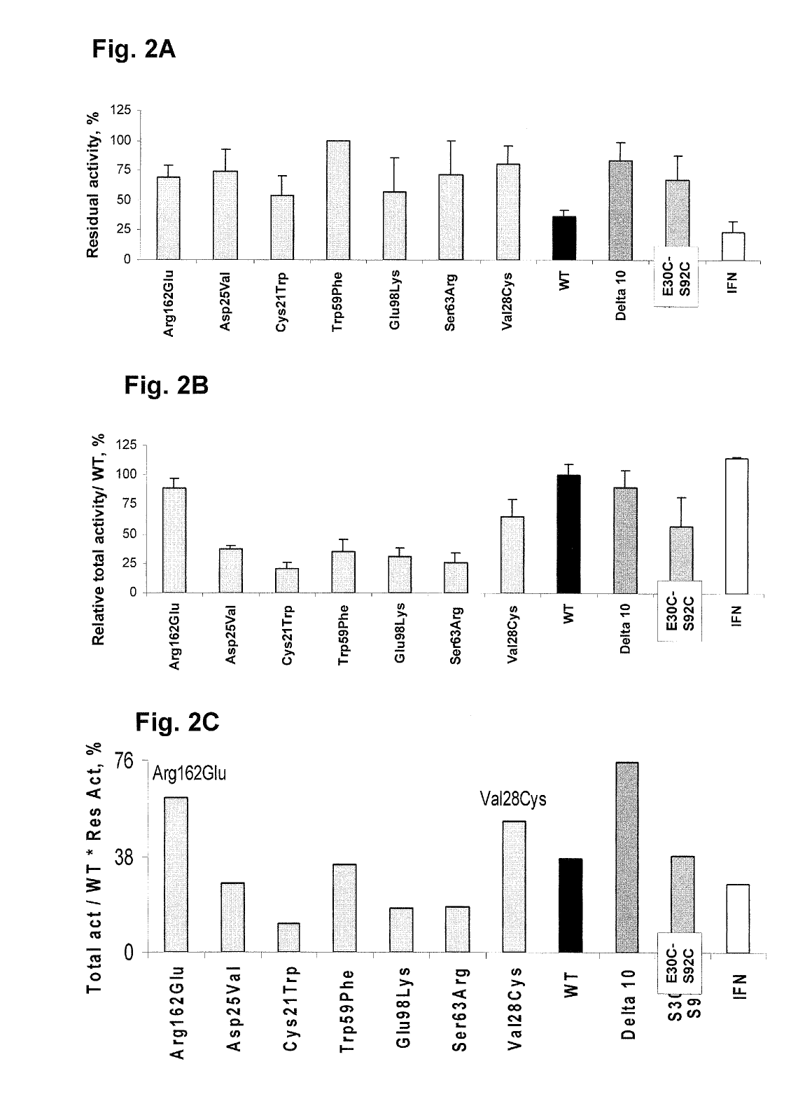

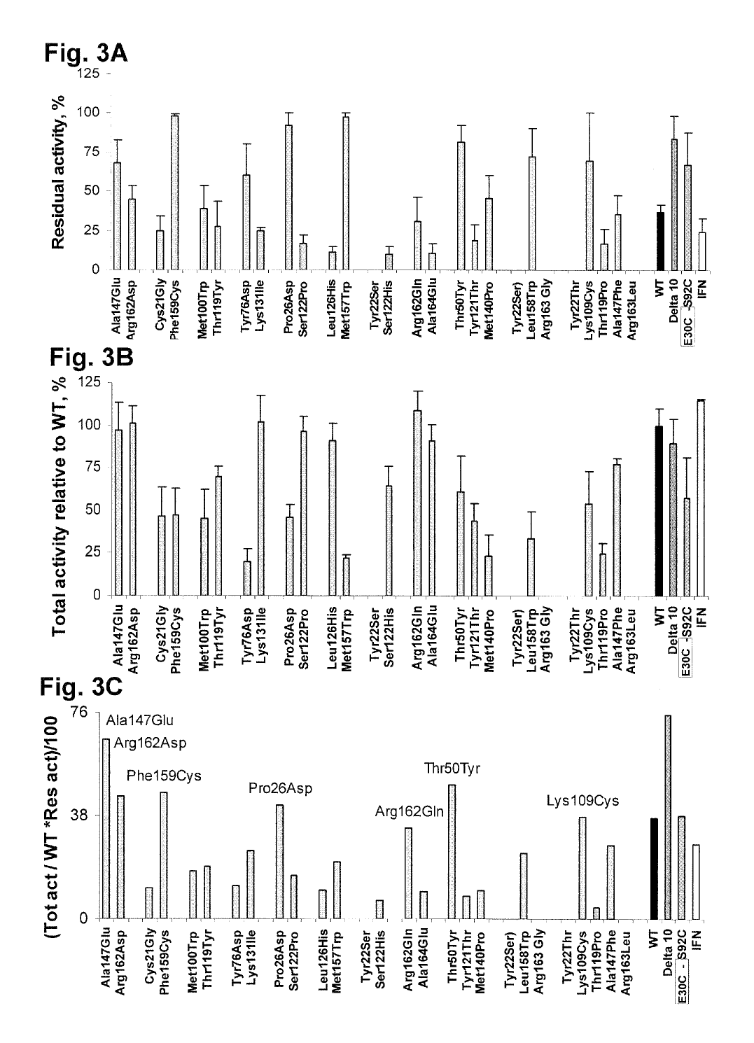

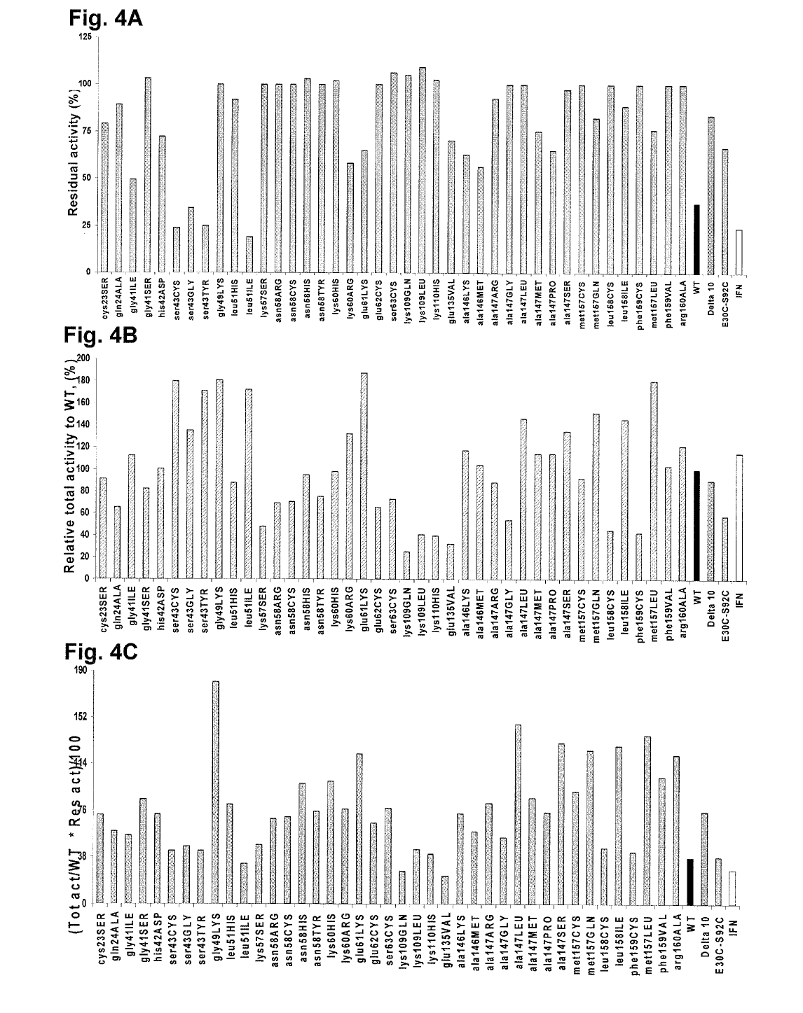

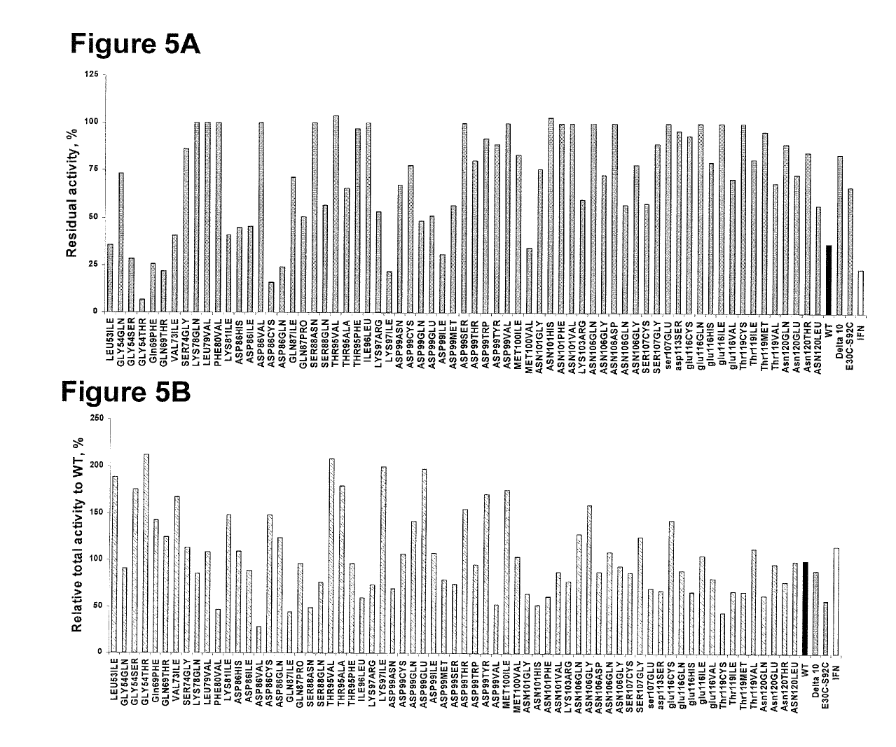

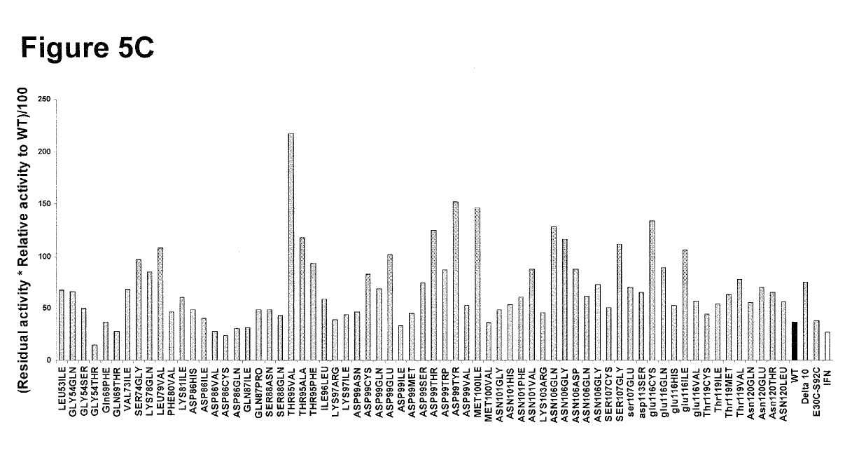

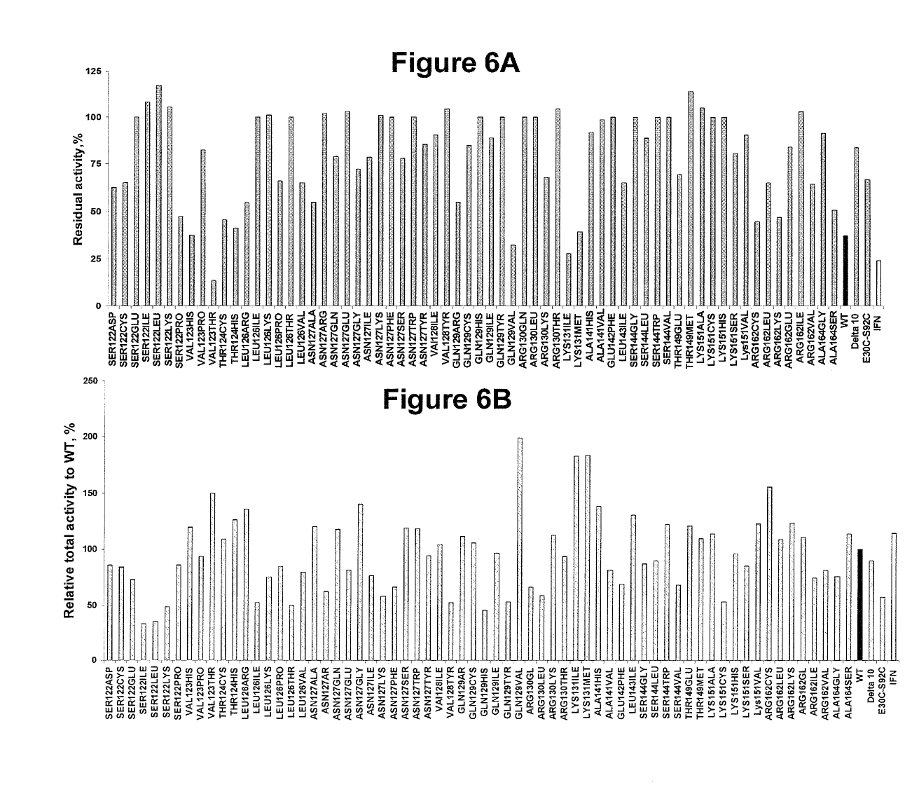

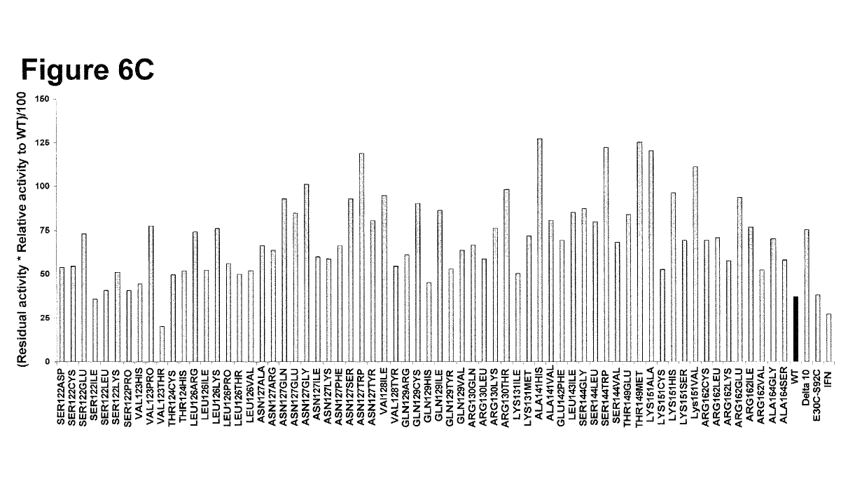

The present invention relates to a thermostable variant of human IFNγ or a functional fragment thereof comprising at least one substitution described in Table 1, Table 2 and FIGS. 2 to 9.

The invention preferably relates to a thermostable variant of human IFNγ or a functional fragment thereof comprising at least one substitution selected in one of the groups consisting of:

(a) C21G, C21W, Y22D, Y22T, Y22S, S23C, Q24A, D25V, P26D, V28C, G41I, G41S, H42D, S43C, S43G, S43T, G49K, T50Y, L51H, L51I, L53I, G54Q, G54S, G54T, K57S, N58R, N58C, N58H, N58Y, W59F, K60H, K60R, E61K, E62C, S63R, S63C, Q69F, Q69T, V73I, S74G, Y76D, K78Q, L79V, F80V, K81I, D86C, D86Q, D86H, D86I, D86V, Q87I, Q87P, S88N, S88Q, T95A, I95V, T95F, 196L, K97I, K97R, E98K, D99N, D99C, D99Q, D99E, D99I, D99M, D99S, D99T, D99W, D99Y, D99V, M100I, M100V, M100N, N101G, N101H, N101F, N101V, K103R, N106D, N106Q, N106G, S107C, S107G, S107E, K109C, K109Q, K109L, K110H, D113S, E116C, E116Q, E116H, E116I, E116V, T119C, T119I, T119M, T119V, T119Y, T119P, N120Q, N120E, N120L, N120T, Y121T, S122D, S122C, S122E, S122I, S122L, S122K, S122P, S122H, V123T, V123H, V123P, T124C, T124H, L126R, L1261, L126K, L126P, L126T, L126V, N127A, N127R, N127Q, N127E, N127G, N127I, N127K, N127F, N127S, N127W, N127Y, V128I, V128Y, Q129R, Q129C, Q129H, Q129I, Q129Y, Q129V, R130Q, R130L, R130K, R130T, K131M, K131I, E135V, M140P, A141H, A141V, E142F, L143I, S144G, S144L, S144W, S144V, A146K, A146M, A147R, A147G, A147E, A147F, A147L, A147M, A147P, A147S, T149E, T149M, K151A, K151C, K151H, K151S, K151V, M157Q, M157W, M157L, L158W, L158C, L158I, F159C, F159V, R160A, R162D, R162E, R162Q, R162C, R1621, R162L, R162K, R162V, R163T, R163L, R163G, A164G, A164S, A164E et S165V or a combination thereof; or
(b) C21G, C21W, Y22D, Y22T, Y22S, Q24A, D25V, P26D, V28C, G41I, G41S, H42D, S43C, S43G, S43T, G49K, T50Y, L51H, L51I, K57S, N58R, N58C, N58H, N58Y, W59F, K60H, K60R, E61K, E62C, S63R, S63C, Y76D, E98K, M100N, K109C, K109Q, K109L, K110H, T119Y, T119P, Y121T, S122H, S122P, K131I, E135V, M140P, A146K, A146M, A147R, A147G, A147E, A147F, A147L, A147M, A147P, A147S, M157Q, M157W, M157L, L158W, L158C, L158I, F159C, F159V, R160A, R162D, R162E, R162Q, R163T, R163L, R163G, A164E et S165V or a combination thereof; or
(c) L53I, G54Q, G54S, G54T, Q69F, Q69T, V73I, S74G, K78Q, L79V, F80V, K81I, D86C, D86Q, D86H, D86I, D86V, Q87I, Q87P, S88N, S88Q, T95A, T95V, T95F, I96L, K97I, K97R, D99N, D99C, D99Q, D99E, D99I, D99M, D99S, D99T, D99W, D99Y, D99V, M100I, M100V, N101G, N101H, N101F, N101V, K103R, N106D, N106Q, N106G, S107C, S107G, S107E, D113S, E116C, E116Q, E116H, E116I, E116V, T119C, T119I, T119M, T119V, N120Q, N120E, N120L, N120T, S122D, S122C, S122E, S1221, S122L, S122K, S122P, V123T, V123H, V123P, T124C, T124H, L126R, L1261, L126K, L126P, L126T, L126V, N127A, N127R, N127Q, N127E, N127G, N127I, N127K, N127F, N127S, N127W, N127Y, V128I, V128Y, Q129R, Q129C, Q12911, Q129I, Q129Y, Q129V, R130Q, R130L, R130K, R130T, K131I, K131M, A141H, A141V, E142F, L143I, S1440, S144L, S144W, S144V, T149E, T149M, K151A, K151C, K151H, K151S, K151V, R162C, R162E, R1621, R162L, R162K, R162V, A164G, A164S or a combination thereof; or

### (d) S63C, E62C, F159C, D99Y, E116C, L158C, S74G, R162C, S122D, L126P, N58R, and T95V.

“Comprise” will be understood to mean that the variant or the fragment thereof has one or more substitutions such as indicated with reference to polypeptide sequences SEQ ID Nos. 2, 4 and 6, but that it may have other modifications, in particular substitutions, deletions or additions.

“Have” will be understood to mean that the variant or fragment thereof contains only the substitution(s) indicated with reference to polypeptide sequences SEQ ID Nos. 2, 4 and 6.

The present invention relates to a thermostable variant of human IFNγ or a functional fragment thereof comprising a combination of substitutions selected from the aforementioned groups. The combination may consist of 2, 3, 4, 5, 6, 7, 8, 9 or 10 substitutions selected from said group. Moreover, the thermostable human IFNγ variant according to the invention may comprise other mutations not described in said group, preferably substitutions, in particular certain known in the field. By way of illustration, the known substitutions are described in WO 92/08737, WO 99/03887, WO01/36001, WO02/81507, WO03/002152, WO2004/005341, WO2004/022593, U.S. Pat. No. 6,958,388, U.S. Pat. No. 4,898,931, U.S. Pat. No. 6,046,034 and WO2006/120580.

For example, the present invention relates to a thermostable variant of human IFNγ or a functional fragment thereof comprising at least one substitution selected in the group consisting of C21W, Q24A, D25V, P26D, V28C, G41I, G41S, H42D, G49K, T50Y, L51H, K57S, N58R, N58C, N58H, N58Y, W59F, K60H, K60R, E61K, E62C, S63R, S63C, Y76D, E98K, K109C, K109L, K109Q, K110H, E135V, M140P, A146K, A146M, A147R, A147G, A147L, A147M, A147P, A147S, A147E, M157W, M157Q, M157L, L158C, L158I, L158W, F159C, F159V, R160A, R162D, R162Q and R162E or a combination thereof. In another example, the present invention relates to a thermostable variant of human IFNγ or a functional fragment thereof comprising at least one of the mutations selected in the group consisting of Q24A, P26D, V28C, G41I, G41S, H42D, G49K, T50Y, L51H, K57S, N58R, N58C, N581-1, N58Y, K60H, K60R, E61K, E62C, S63C, K109C, A146K, A146M, A147R, A147G, A147L, A147M, A147P, A147S, A147E, M157Q, M157L, L158I, F159C, F159V, R160A, R162E. R162Q and R162D or a combination thereof.

In a still more preferred embodiment, the present invention relates to a thermostable variant of human IFNγ or a functional fragment thereof comprising at least a first substitution selected in the group consisting of S63C, E62C, F159C, D99Y, E116C, L158C, S74G, R162C, S122D, L126P, N58R, and T95V. In a preferred embodiment, the variant does not contain any non-peptide moiety attached to the residue(s) introduced by said first substitution(s). Said variant may additionally comprise at least one other substitution selected in the group consisting of M157C, G41S and M100N.

In another embodiment, the present invention also relates to a thermostable variant of human IFNγ or a functional fragment thereof having either a single substitution C23 S or M157C, or a substitution C23S or M157C in combination with one or more substitutions selected in the group consisting of C21G, C21W, Y22D, Y22T, Y22S, Q24A, D25V, P26D, V28C, G41I, G41S, H42D, S43C, S43G, S43T, G49K, T50Y, L51H, L51I, K57S, N58R, N58C, N58H, N58Y, W59F, K60H, K60R, E61K, E62C, S63R, S63C, Y76D, E98K, M100N, K109C, K109Q, K109L, K110H, T119Y, T119P, Y121T, S122H, S122P, K131I, E135V, M140P, A146K, A146M, A147R, A147G, A147E, A147F, A147L, A147M, A147R, A147S, M157Q, M157L, L158W, L158C, L158I, F159C, F159V, R160A, R162D, R162E, R162Q, R163T, R163L, R163G, A164E, S165V, L53I, G54Q, G54S, G54T, Q69F, Q69T, V73I, S74G, K78Q, L79V, F80V, K81I, D86C, D86Q, D86H, D86I, D86V, Q87I, Q87P, S88N, S88Q, T95A, T95V, T95F, 196L, K97I, K97R, D99N, D99C, D99Q, D99E, D99I, D99M, D99S, D99T, D99W, D99Y, D99V, M100I, M100V, N101G, N101H, N101F, N101V, K103R, N106D, N106Q, N106G, S107C, S107G, S107E, D113S, E116C, E116Q, E116H, E116I, E116V, T119C, T119I, T119M, T119V, N120Q, N120E, N120L, N120T, S122D, S122C, S122E, S122I, S122L, S122K, S122P, V123T, V123H, V123P, T124C, T124H, L126R, L1261, L126K, L126P, L126T, L126V, N127A, N127R, N127Q, N127E, N127G, N127I, N127K, N127F, N127S, N127W, N127Y, V128I, V128Y, Q129R, Q129C, Q129H, Q129I, Q129Y, Q129V, R130Q, R130L, R130K, R130T, K131I, K131M, A141H, A141V, E142F, L143I, S144G, S144L, S144W, S144V, T149E, T149M, K151A, K151C, K151H, K151S, K151V, R162C, R162E, R1621, R162L, R162K, R162V, A164G and A164S. In a preferred embodiment, the present invention relates to a thermostable variant of human IFNγ or a functional fragment thereof having either a single substitution C23S or M157C, or a substitution C23S or M157C in combination with one or more substitutions selected in the group consisting of S63C, E62C, F159C, D99Y, E116C, L158C, S74G, R162C, S122D, L126P, N58R, M100N, T95V and G41S. In a still more preferred embodiment, the present invention relates to a thermostable variant of human IFNγ or a functional fragment thereof having either a single substitution M157C, or a substitution M157C in combination with one or more substitutions selected in the group consisting of S63C, E62C, F159C, D99Y, E116C, L158C, S74G, R162C, S122D, L126P, N58R, M100N, T95V and G41S.

In one embodiment, the present invention relates to a thermostable variant of human IFNγ or a functional fragment thereof having a single substitution selected in one of the groups consisting of:

(a) C21G, C21W, Y22D, Y22T, Y22S, C23S, Q24A, D25V, P26D, V28C, G41I, G41S, H42D, S43C, S43G, S43T, G49K, T50Y, L51H, L51I, K57S, N58R, N58C, N58H, N58Y, W59F, K60H, K60R, E61K, E62C, S63R, S63C, Y76D, E98K, M100N, K109C, K109Q, K109L, K110H, T119Y, T119P, Y121T, S122H, S122P, K131I, E135V, M140P, A146K, A146M, A147R, A147G, A147E, A147F, A147L, A147M, A147P, A147S, M157Q, M157W, M157L, M157C, L158W, L158C, L158I, F159C, F159V, R160A, R162D, R162E, R162Q, R163T, R163L, R163G, A164E and S165V; or
(b) L53I, G54Q, G54S, 054T, Q69F, Q69T, V73I, S74G, K78Q, L79V, F80V, K81I, D86C, D86Q, D86H, D861, D86V, Q87I, Q87P, S88N, S88Q, T95A, T95V, T95F, 196L, K97I, K97R, D99N, D99C, D99Q, D99E, D99I, D99M, D99S, D99T, D99W, D99Y, D99V, M100I, M100V, N101G, N101H, N101F, N101V, K103R, N106D, N106Q, N106G, S107C, S107G, S107E, D113S, E116C, E116Q, E116H, E116I, E116V, T119C, T119I, T119M, T119V, N120Q, N120E, N120L, N120T, S122D, S122C, S122E, S1221, S122L, S122K, S122P, V123T, V123H, V123P, T124C, T124H, L126R, L1261, L126K, L126P, L126T, L126V, N127A, N127R, N127Q, N127E, N127G, N127I, N127K, N127F, N127S, N127W, N127Y, V128I, V128Y, Q129R, Q129C, Q129H, Q129I, Q129Y, Q129V, R130Q, R130L, R130K, R130T, K131I, K131M, A141H, A141V, E142F, L143I, S144G, S144L, S144W, S144V, T149E, T149M, K151A, K151C, K151H, K151S, K151V, R162C, R162E, R1621, R162L, R162K, R162V, A164G and A164S; or

### (c) S63C, E62C, F159C, D99Y, E116C, L158C, S74G, R162C, S122D, L126P, N58R, and T95V.

For example, the substitution is selected in the group consisting of C21W, C23S, Q24A, D25V, P26D, V28C, G41I, G41S, I-142D, G49K, T50Y, L51H, K57S, N58R, N58C, N58H, N58Y, W59F, K60H, K60R, E61K, E62C, S63R, S63C, Y76D, E98K, K109C, K109L, K109Q, K110H, E135V, M140P, A146K, A146M, A147R, A147G, A147L, A147M, A147P, A147S, A147E, M157W, M157Q, M157C, M157L, L158C, L158I, L158W, F159C, F159V, R160A, R162D, R162Q and R162E. In particular, the substitution may be selected in the group consisting of C23S, Q24A, P26D, V28C, G41I, G41S, H42D, G49K, T50Y, L51H, K57S, N58R, N58C, N58H, N58Y, K60H, K60R, E61K, E62C, S63C, K109C, A146K, A146M, A147R, A147G, A147L, A147M, A147P, A147S, A147E, M157Q, M157C, M157L, L158I, F159C, F159V, R160A, R162E, R162Q and R162D. Preferably, the present invention relates to a thermostable variant of human IFNγ or a functional fragment thereof having a single substitution selected in the group consisting of S63C, E62C, F159C, D99Y, E116C, L158C, S74G, R162C, S122D, L126P, N58R, and T95V.

In one embodiment, the present invention relates to a thermostable variant of human IFNγ or a functional fragment thereof having a combination of substitutions selected in the group consisting of C21G+F159C, A147E+R162D, M100N+T119Y, Y76D+K131I, T50Y+Y121T+M140P, P26D+S122P, Y22S+L158W+R163G, Y22D+S122H, R162Q+A164E, and Y22T+K109C+T119P+A147F+R163L.

In another embodiment, the present invention relates to a thermostable variant of human IFNγ or a functional fragment thereof comprising or having a combination of two substitutions selected in the group consisting of S63C, E62C, F159C, D99Y, E116C, L158C, S74G, R162C, S122D, M100N, L126P, N58R, T95V, M157C and G41S, preferably a combination selected in the group consisting of S63C+E62C, S63C+F159C, S63C+D99Y, S63C+E116C, S63C+L158C, S63C+S74G, S63C+R162C, S63C+S122D, S63C+M100N, S63C+L126P, S63C+N58R, S63C+T95V, S63C+M157C, S63C+G41S, E62C+F159C, E62C+D99Y, E62C+E116C, E62C+L158C, E62C+S74G, E62C+R162C, E62C+S122D, E62C+M100N, E62C+L126P, E62C+N58R, E62C+T95V, E62C+M157C, E62C+G41S, F159C+D99Y, F159C+E116C, F159C+L158C, F159C+S74G, F159C+R162C, F159C+S122D, F159C+M100N, F159C+L126P, F159C+N58R, F159C+T95V, F159C+M157C, F159C+G41S, D99Y+E116C, D99Y+L158C, D99Y+S74G, D99Y+R162C, D99Y+S122D, D99Y+M100N, D99Y+L126P, D99Y+N58R, D99Y+T95V, D99Y+M157C, D99Y+G41S, E116C+L158C, E116C+S74G, E116C+R162C, E116C+S122D, E116C+M100N, E116C+L126P, E116C+N58R, E116C+T95V, E116C+M157C, E116C+G41S, L158C+S74G, L158C+R162C, L158C+S122D, L158C+M100N, L158C+L126P, L158C+N58R, L158C+T95V, L158C+M157C, L158C+G41S, S74G+R162C, S74G+S122D, S74G+M100N, S74G+L126P, S74G+N58R, S74G+T95V, S74G+M157C, S74G+G41S, R162C+S122D, R162C+M100N, R162C+L126P, R162C+N58R, R162C+T95V, R162C+M157C, R162C+G41S, S122D+L126P, S122D+N58R, S122D+T95V, S122D+M157C, S122D+M100N, S122D+G41S, L126P+N58R, L126P+T95V, L126P+M157C, L126P+M100N, L126P+G41S, N58R T95V, N58R+M157C, N58R+M100N, N58R+G41S, T95V+M157C, T95V+M100N, T95V+G41S, M157C+M100N and M157C+G41S. In a preferred embodiment, the present invention relates to a variant comprising or having a combination of substitutions selected in the group consisting of S63C+E62C, S63C+F159C, S63C+D99Y, S63C+E116C, S63C+L158C, S63C+S74G, S63C+R162C, S63C+S122D, S63C+M100N, S63C+L126P, S63C+N58R, S63C+T95V, S63C+M157C, S63C+G41S, preferably the combination S63C+G41S.

The sequences SEQ ID Nos. 1 to 6 describe the protein sequences of the precursor and mature forms of human IFNγ and the nucleic acid sequences encoding them. In a preferred embodiment, the variant according to the present invention corresponds to the precursor protein of 166 amino acid residues (SEQ ID No. 2), or to the mature protein with or without the CYC tripeptide (SEQ ID Nos. 6 and 4, respectively) comprising at least one substitution or a combination of substitutions according to the invention. In particular, the term “variant” denotes a polypeptide differing from a polypeptide having a sequence selected from sequences SEQ ID Nos. 2, 4 and 6 by at least 1, 2, 3, 4, 5, 6, 7, 8, 9 or 10 residue(s), preferably by 1, 2, 3, 4, 5, 6, 7, 8, 9 or 10 residue(s).

“Functional fragment” denotes a fragment of human IFNγ having the activity of human IFNγ. For example, said fragment may correspond to the precursor or mature form of human IFNγ, with or without the CYC tripeptide, with a C-terminal deletion of 1, 2, 3, 4, 5, 6, 7, 8, 9, 10, 11, 12, 13, 14, 15, 16, 17, 18, 19 or 20 amino acid residues, preferably from 1 to 15 residues, even more preferably from 1 to 11 residues. The fragment may comprise 100, 110, 120, 130 or 140 consecutive amino acid residues of human IFNγ.

“Human IFNγ activity” refers to the capability to bind to the human IFNγ receptor and to cause transduction of the signal induced upon human IFNγ binding to its receptor as determined in vitro or in vivo. Human IFNγ activity may be measured by the methods described below in the description and in the examples.

The variants according to the present invention have increased thermostability as compared to wild-type human IFNγ. Said increase is at least 5%, preferably at least 10, or 30%. “Thermostability” will be understood to mean the ability of the protein to conserve its activity after being exposed to the action of heat. For example, the protein can be incubated for 10 minutes at 59° C. The thermostability of the variant is then evaluated by the percentage of residual activity after said pretreatment. This measure of the thermostability of a variant is then compared to the same value obtained by using wild-type IFNγ subjected to the same conditions.

A variant which has improved thermostabilty but reduced activity may be used. Preferably, the thermostable variants of the invention conserve an activity (condition without pretreatment) which corresponds to at least 10% of the activity of wild-type human IFNγ, preferably at least 20, 30, 40, 50, 60, 70, 80 or 90% of the activity of wild-type human IFNγ. In a particularly preferred embodiment, the thermostable variants of the invention conserve an activity which is equivalent, or even higher than, that of wild-type human IFNγ.

An interesting factor for selecting variants of interest is the relative activity of the variant multiplied by the percentage of residual activity.

“Non-peptide moiety” is intended to indicate a non-peptide molecule which can be attached to the side chain of an amino acid of human IFNγ. Said molecule can be a polymer molecule, a lipophilic molecule, a carbohydrate or an organic derivatizing agent. The carbohydrate can be attached to IFNγ by in vitro or in vivo glycosylation, for example by N- or O-glycosylation. A lipophilic molecule can be for example a saturated or unsaturated fatty acid, a terpene, a vitamin, a steroid or carotenoid. A polymer molecule can be a polyol, a polyamine, a polycarbocyclic acid or a polyalkylene oxide, in particular a polyethylene glycol (PEG). This type of molecule is well known to those skilled in the art. A variant containing a PEG group will be referred to as being “PEGylated”.

The human IFNγ variant according to the present invention can be glycosylated, preferably at positions 48 and 120. In another embodiment, the variant can be not glycosylated. When the variant comprises the substitution G41S, it can be glycosylated at position N39 by N-glycosylation. In an alternative embodiment, such variant can be not glycosylated at this position.

In one embodiment, the variant can be modified by adding a polymer molecule, in particular by adding polymers (Kita et al., Drug Des. Deliv. 6:157-167, 1990; EP 236987 and U.S. Pat. No. 5,109,120) or by pegylation (WO99/03887; see WO2004005341 “Conjugation of a polymer molecule”). In a preferred embodiment of a human IFNγ variant according to the invention, the polymer molecule is attached at a position other than positions 41, 58, 62, 63, 74, 95, 99, 100, 116, 122, 126, 157, 158, 159 and 162. In particular, the molecule is not attached to a residue introduced by a substitution made in a variant according to the invention, in particular a substitution selected in the group consisting of S63C, E62C, F159C, D99Y, M100N, E116C, L158C, S74G, R162C, S122D, L126P, N58R, T95V, G41S and M157C.

In another embodiment, the variant according to the invention does not contain any polymer molecule. In particular, it is not pegylated.

The in vitro activity of IFNγ is generally measured in terms of the reduction of the cytopathic effect of a virus on a cell line after treatment with increasing amounts of IFNγ. Other biological tests specific for IFNγ exist (Meager, J. Immunol. Methods, 261, (2002) 21-36 for a review). In particular these new tests make it possible to more specifically evaluate one of the characteristics of the pleiotropic activity of IFNγ. Tests of in vivo activity also exist.

The antiviral response to IFNγ can be measured in different coupled systems (virus/IFNγ-responsive adherent cell line sensitive to the virus used). Examples include the following coupled systems: VSV/MDBK; VSV or EMCV/A549; VSV, EMCV, SFV or Sindbis virus/WISH; VSV or EMCV/HeLa; VSV, EMCV or Mengovirus/FS4, FS71, or Hep2; VSV, EMCV, Mengovirus or Sindbis virus/FL; EMCV/2D9, where VSV=vesicular stomatitis virus; EMCV=encephalomyocarditis virus; SFV=Semliki forest virus. Preferably, the used viruses will be the vaccinia virus or the lymphocytic choriomeningitis virus (LCMV). The herpex simplex virus (HSV) and the cytomegalovirus may also be used.

The activity of IFNγ can also be tested by using a reporter gene, for example luciferase, under the control of an IFNγ-responsive promoter containing GAS (gamma-interferon activation sites) or ISRE elements (interferon stimulated response element). Thus, the reporter gene is assayed after stimulation by IFNγ. The pGAS/Luciferase and pISRE/Luciferase vectors are commercially available (#219091, Stratagene). In a preferred embodiment, IFNγ activity is measured by the method using the pGAS/luciferase vector.

The increase in the thermostability of IFNγ all while conserving its biological activity makes it possible to envision the development of more effective treatments which, for an equivalent biological activity, enable a reduction in the therapeutic doses used and a resultant reduction in the adverse effects of the treatment. This would also allow treatments using higher doses of IFNγ in order to reduce viral infections such as herpes, said treatments heretofore being inconceivable with this type of molecule. Moreover, the variants according to the present invention may have the advantage of a longer half-life during their storage, and therefore a better shelf-life, as compared to wild-type IFNγ, in particular at room temperature.

The present invention therefore relates to a pharmaceutical composition comprising a thermostable IFNγ variant according to the invention.

Thus, the present invention preferably relates to a pharmaceutical composition comprising a thermostable IFNγ variant or a functional fragment thereof comprising at least one substitution selected in the group consisting of S63C, E62C, F159C, D99Y, E116C, L158C, S74G, R162C, S122D, L126P, N58R, and T95V, the variant not containing a non-peptide moiety attached to the residue(s) introduced by the first substitution(s). In one embodiment, the variant differs from a polypeptide having a sequence selected from among the sequences SEQ ID Nos. 2, 4 and 6 by at least 1, 2, 3, 4, 5, 6, 7, 8, 9 or 10 residue(s), preferably by 1, 2, 3, 4, 5, 6, 7, 8, 9 or 10 residue(s). In a particular embodiment, the variant has a single substitution. In another embodiment, the variant additionally comprises at least one other substitution selected in the group consisting of M157C, G41S and M100N. In particular, the variant may comprise or have a combination of two substitutions selected in the group consisting of S63C, E62C, F159C, D99Y, E116C, L158C, S74G, R162C, S122D, M100N, L126P, N58R, T95V, M157C and G41S. In a preferred embodiment, the variant comprises or has a combination of substitutions selected in the group consisting of S63C+E62C, S63C+F159C, S63C+D99Y, S63C+E116C, S63C+L158C, S63C+S74G, S63C+R162C, S63C+S122D, S63C+M100N, S63C+L126P, S63C+N58R, S63C+T95V, S63C+M157C, S63C+G41S, E62C+F159C, E62C+D99Y, E62C+E116C, E62C+L158C, E62C+S74G, E62C+R162C, E62C+S122D, E62C+M100N, E62C+L126P, E62C+N58R, E62C+T95V, E62C+M157C, E62C+G41S, F159C+D99Y, F159C+E116C, F159C+L158C, F159C+S74G, F159C+R162C, F159C+S122D, F159C+M100N, F159C+L126P, F159C+N58R, F159C+T95V, F159C+M157C, F159C+G41S, D99Y+E116C, D99Y+L158C, D99Y+S74G, D99Y+R162C, D99Y+S122D, D99Y+M100N, D99Y+L126P, D99Y+N58R, D99Y+T95V, D99Y+M157C, D99Y+G41S, E116C+L158C, E116C+S74G, E116C+R162C, E116C+S122D, E116C+M100N, E116C+L126P, E116C+N58R, E116C+T95V, E116C+M157C, E116C+G41S, L158C+S74G, L158C+R162C, L158C+S122D, L158C+M100N, L158C+L126P, L158C+N58R, L158C+T95V, L158C+M157C, L158C+G41S, S74G+R162C, S74G+S122D, S74G+M100N, S74G+L126P, S74G+N58R, S74G+T95V, S74G+M157C, S74G+G41S, R162C+S122D, R162C+M100N, R162C+L126P, R162C+N58R, R162C+T95V, R162C+M157C, R162C+G41S, S122D+L126P, S122D+N58R, S122D+T95V, S122D+M157C, S122D+M100N, S122D+G41S, L126P+N58R, L126P+T95V, L126P+M157C, L126P+M100N, L126P+G41S, N58R+T95V, N58R+M157C, N58R+M100N, N58R+G41S, T95V+M157C, T95V+M100N, T95V+G41S, M157C+M100N and M157C+G41S. In a still more preferred embodiment, the variant comprises or has a combination of substitutions selected in the group consisting of S63C+E62C, S63C+F159C, S63C+D99Y, S63C+E116C, S63C+L158C, S63C+S74G, S63C+R162C, S63C+S122D, S63C+M100N, S63C+L126P, S63C+N58R, S63C+T95V, S63C+M157C, S63C+G41S. In a particularly preferred embodiment, the variant comprises or has the combination S63C+G41S. In a particular embodiment, the variant does not have a deletion of 1 to 11 residues at the C-terminal end. In another particular embodiment, the variant has a deletion of 1 to 11 residues at the C-terminal end. In a particular embodiment, the variant does not contain any non-peptide moiety selected in the group consisting of a polymer molecule, a lipophilic molecule, and an organic derivatizing agent. In another particular embodiment, the variant does contain a non-peptide moiety selected in the group consisting of a polymer molecule, a lipophilic molecule, and an organic derivatizing agent. The non-peptide moiety in question is more particularly a polymer molecule, preferably a polyethylene glycol. In a particular embodiment, the variant is glycosylated. In particular the variant can be glycosylated at position N39 by an N-glycosylation when it comprises the substitution G41S.

A pharmaceutical composition according to the invention may additionally comprise a pharmaceutically acceptable support or excipient. Said supports and excipients are well known to those skilled in the art (Remington's Pharmaceutical Sciences, 18th edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]).

A pharmaceutical composition according to the invention may be formulated in a variety of forms, in particular liquid, gel, lyophilised, powder, compressed solid, and other forms.

The present invention further concerns a thermostable IFNγ variant according to the invention or a composition according to the invention as medicament.

The pharmaceutical compositions of the invention are suitable or formulated for oral, parenteral (intradermal, intramuscular, intravenous, subcutaneous), sublingual, topical, local, intratracheal, intranasal, transdermal, rectal, intraocular, intraauricular administration, said active agent being able to be administered in the form of a dosage unit.

An example of a pharmaceutical composition is a solution for parenteral administration. Although the composition may be in liquid form, appropriate for immediate use, such parenteral forms may also be provided in frozen or lyophilised form. In this case, the composition will be thawed or reconstituted prior to use. In the case of lyophilisation, the composition will be prepared by the addition of a pharmaceutically acceptable diluent such as sterile water for injection or sterile physiological saline solution.

Dosage unit forms may for example be tablets, capsules, gels, granules, powders, oral or injectable solutions or suspensions, transdermal patches, sublingual, buccal, intratracheal, intraocular, intranasal, intraauricular forms, inhalation, topical, transdermal, subcutaneous, intramuscular or intravenous forms, rectal administration forms or implants.

Formulations for topical administration include creams, gels, ointments, lotions or drops. Said pharmaceutical formulations are prepared according to the conventional methods of the relevant field. In a preferrred embodiment, the pharmaceutical composition is liquid.

Said dosage unit forms are dosed so as to enable a daily administration of 0.001 to 100 μg of active ingredient per kg of body weight, depending on the pharmaceutical formulation. There may be special cases where higher or lower dosage strengths are appropriate; said dosages do not deviate from the scope of the invention. In accordance with standard practice, the appropriate dose for each patient is determined by the physician according to the method of administration, the weight and the response of the patient.

In a preferred embodiment, IFNγ is administered by the parenteral route, and preferably by subcutaneous injection. For example, a usual subcutaneous injection dose of IFNγ is comprised between 1 and 100 μg/m² if body surface area is greater than 0.5 m² and between 0.01 and 10 μg/kg of body weight if body surface area is less than or equal to 0.5 m².

IFNγ is the archetype of a pleiotropic cytokine with a broad spectrum of activities. In fact, the interferons (IFNs) are endowed with activities such as inhibition of viral replication, inhibition of cell multiplication and induction of apoptosis.

In particular, stimulation of macrophages by IFNγ induces the following responses:
increase in phagocytosis and bacterial killing (direct antimicrobial and antitumoral mechanisms);
stimulation of antigen presentation and degradation pathways, expression of major histocompatibility complex (MHC) type I and II at the macrophage surface;

differentiation of B lymphocytes to antibody-secreting plasmocytes, which results in immunoglobulin IgG production and complement activation;
activation of NO synthase giving rise to production of cytotoxic oxygen free radicals and NO; and/or
increase in the production of cytokines and endogenous IFN.

IFNγ acts on T lymphocytes by promoting their differentiation thereby modulating the specific immune response.

Among the pharmacological properties of IFNγ, the main effect sought after and developed in clinical phases is primarily the immunomodulating aspect, the antiviral therapeutic molecule aspect being less extensively developed up to now.

By virture of its broad spectrum of activities (antiviral, antiproliferative and immunomodulating), IFNγ has been developed as a therapeutic agent in the treatment of a wide variety of human diseases. Commercial IFNγ (Actimmune, and Biogamma) are used in particular for two main therapeutic indications: chronic granulomatous disease and idiopathic pulmonary fibrosis, in combination with oral prednisolone. In addition to the main therapeutic indications, many novel secondary indications are currently in different clinical phases of development (II and III), in particular for their immunosuppressive role for example as an adjunct to pegylated IFNα/ribavirin in the treatment of hepatitis C. Atypical mycobacterial infections; kidney cancer; osteopetrosis; systemic scleroderma; chronic hepatitis B; chronic hepatitis C; septic shock; allergic dermatitis; rheumatoid arthritis; ovarian cancer; hepatic fibrosis; asthma; and lymphoma may also be mentioned.

IFNγ is also useful in the treatment of various viral infections, and displays activity against human papillomavirus infection and hepatitis B and C viral infection.

Moreover, if the problem of toxicity associated with high doses of IFNγ were to be diminished or resolved by a more stable molecule, and therefore one having a stronger effect in vivo, the use of IFNγ in novel indications, and in particular for treating herpes (HSV) type I and II viral infections might be reconsidered.

Thus, the present invention concerns the use of a thermostable IFNγ variant according to the invention or a pharmaceutical composition according to the invention for preparing an antiviral, antiproliferative or immunomodulating medicament. For instance, said medicament is designed for treating inflammatory diseases, cancers, infections, bone disorders, autoimmune disorders, and the like. In a preferred embodiment, the medicament is designed for treating a pathology selected in the group consisting of asthma, chronic familial granulomatous disease, idiopathic pulmonary fibrosis, atypical mycobacterial infection, kidney cancer, osteopetrosis, systemic scleroderma, chronic hepatitis B or C, septic shock, allergic dermatitis, and rheumatoid arthritis. In alternative embodiments, the medicament is designed for treating a pathology selected in the group consisting of prurigo, neurodermatitis, type 1 diabetes, vascular stenosis, basocellular epithelioma, a cancer or lymphoma such as ovarian cancer, kidney cancer, a leukemia such as a B or T cell proliferative disorder, chronic myeloid leukemia and related syndromes, breast cancer, lung cancer, melanoma, colorectal cancer, brain cancer, pleural cancer, stomach cancer, pancreatic cancer, a viral infection, for example due to hepatitis B or C virus, Crohn's disease, psoriasis, multiple sclerosis, and amyotrophic lateral sclerosis.

The thermostable IFNγ variant according to the present invention may be used in combination with another active agent, for example an active agent selected in the group consisting of an antibody, an antitumoral or chemotherapeutic agent, a glucocorticoid, an antihistamine, an adrenocortical hormone, an anti-allergic agent, a vaccine, a bronchodilator, a steroid, a beta-adrenergic agent, an immunomodulating agent, a cytokine such as interferon alpha or beta, interleukin 1 or 2, TNF (tumor necrosis factor), hydroxyurea, an alkylating agent, a folic acid antagonist, a nucleic acid antimetabolite, a spindle poison, an antibiotic, a nucleotide analogue, a retinoid, a lipoxygenase and cyclooxygenase inhibitor, fumaric acid and its salts, an analgesic, a spasmolytic, a calcium antagonist and a combination thereof. The additional active agent may be administered prior to, concurrently with, or after administration of IFNγ according to the invention. Furthermore, it may be administered by the same route of administration or by two different routes of administration. Thus, the present invention concerns a product comprising a thermostable IFNγ variant or a pharmaceutical composition according to the invention and another active agent, preferably selected from the above list, for a combined preparation designed for simultaneous, sequential or separate use for treating one of the aforementioned pathologies.

The combination with an antibody is useful for treating cancer. In fact, IFNγ can increase the effect of antibodies by ADCC (antibody-dependent cellular cytotoxicity). The antibody is preferably directed against an antigen exposed on the cancer cells. The antibody may be a polyclonal, monoclonal, humanised or chimeric antibody. Preferably, the antibody is monoclonal and humanised. For example, in the case of a B cell lymphoproliferative disorder like non-Hodgkin's lymphoma, the antigen can be CD20. Said antibody can be Rituximab.

The combination with a glucocorticoid is useful for treating pulmonary alveolar diseases such as idiopathic pulmonary fibrosis. Examples of suitable glucocorticoids include hydrocortisone, cortisone, dexamethasone, betamethasone, prednisolone, methyl prednisolone and their pharmaceutically acceptable salts. A preferred embodiment of the invention concerns the use of a combination of IFNγ according to the invention and prednisolone.

The combination with an antihistamine, an adrenocortical hormone, an anti-allergic agent is useful in particular for treating skin diseases such as prurigo or neurodermatitis.

The combination with an anti-allergic agent, a bronchodilator, a steroid, a beta-adrenergic agent, an immunomodulating agent, or a cytokine is useful in particular for treating asthma.

The present invention further relates to a method of antiviral, antiproliferative or immunomodulating treatment in a patient requiring same, comprising administering to the patient a therapeutically effective amount of a thermostable IFNγ variant or a pharmaceutical composition according to the invention. Preferably, the method of treatment is designed for treating a pathology mentioned hereinabove. Optionally, the method may additionally comprise administering another active agent, preferably selected from those cited above. A therapeutically effective amount is the amount necessary to reduce or eliminate the symptoms of the disease or to manage or slow the progression of the disease. The patient is preferably human.

The present invention relates to a nucleic acid encoding a thermostable variant of human IFNγ according to the invention. The invention also relates to an expression cassette for a nucleic acid according to the invention. It further relates to a vector comprising a nucleic acid or expression cassette according to the invention. The vector may be selected from a plasmid and a viral vector.

The nucleic acid may be DNA (cDNA or gDNA), RNA, a mixture of the two. It may be single stranded or duplex or a mixture of the two. It may comprise modified nucleotides, comprising for example a modified bond, a modified purine or pyrimidine base, or a modified sugar. It may be prepared by any methods known to those skilled in the art, including chemical synthesis, recombination, mutagenesis, and the like.

The expression cassette comprises all the necessary components for expression of the thermostable human IFNγ variant according to the invention, in particular the components required for transcription and translation in the host cell. The host cell may be prokaryotic or eukaryotic. In particular, the expression cassette comprises a promoter and a terminator, optionally an amplifier. The promoter may be prokaryotic or eukaryotic. Examples of preferred prokaryotic promoters are the following: Lacl, LacZ, pLacT, ptac, pARA, pBAD, RNA polymerase promoters of bacteriophage T3 or T7, the polyhedrin promoter, the PR or PL promoter of phage lambda. Examples of preferred eukaryotic promoters are the following: CMV early promoter, HSV thymidine kinase promoter, SV40 early or late promoter, the mouse metallothionein-L promoter, and the LTR regions of certain retroviruses. Generally, in order to choose a suitable promoter the person of the art may advantageously refer to the work of Sambrook et al. (1989) or else to the techniques described by Fuller et al. (1996; Immunology in Current Protocols in Molecular Biology).

The present invention relates to a vector containing a nucleic acid or an expression cassette encoding a thermostable human IFNγ variant according to the invention. The vector is preferably an expression vector, that is, it comprises the necessary components for expression of the variant in the host cell. The host cell may be a prokaryote, for example E. coli, or a eukaryote. The eukaryote may be a lower eukaryote such as a yeast (for example, S. cerevisiae) or fungus (for example of the genus Aspergillus) or a higher eukaryote such as an insect cell (Sf9 or Sf21 for example), mammalian cell or plant cell. The cell may be a mammalian cell, for example COS (green monkey cell line) [for example, COS 1 (ATCC CRL-1650), COS 7 (ATCC CRL-1651), CHO (U.S. Pat. No. 4,889,803; U.S. Pat. No. 5,047,335, CHO-K1 (ATCC CCL-61)], mouse cells or human cells. In a particular embodiment, the cell is non-human and non-embryonic. The vector may be a plasmid, phage, phagemid, cosmid, virus, YAC, BAC, a pTi plasmid of Agrobacterium, and the like. The vector may preferably comprise one or more elements selected in the group consisting of an origin of replication, a multiple cloning site and a selection gene. In a preferred embodiment, the vector is a plasmid. Non-limiting examples of prokaryotic vectors are the following: pQE70, pQE60, pQE-9 (Qiagen), pbs, pD10, phagescript, psiX174, pbluescript SK, pbsks, pNH8A, pNH16A, pNH18A, pNH46A (Stratagene); ptrc99a, pKK223-3, pKK233-3, pDR540, pBR322, and pRITS (Pharmacia), pET (Novagen). Non-limiting examples of eukaryotic vectors are the following: pWLNEO, pSV2CAT, pPICZ, pcDNA3.1 (+) Hyg (Invitrogen), pOG44, pXT1, pSG (Stratagene); pSVK3, pBPV, pCI-neo (Stratagene), pMSG, pSVL (Pharmacia); and pQE-30 (QLAexpress). Non-limiting examples of viral vectors include adenoviruses, AAV, HSV, lentiviruses, and the like. Preferably, the expression vector is a plasmid or a viral vector.

The sequence encoding IFNγ according to the invention may comprise or not the signal peptide. In the case where it does not comprise it, a methionine may optionally be added at the N-terminal end. In another alternative, a heterologous signal peptide may be introduced. Said heterologous signal peptide may be a derivative of a prokaryote such as E. coli or of a eukaryote, in particular a mammalian, insect or yeast cell.

The present invention relates to the use of a polynucleotide, an expression cassette or a vector according to the invention, for transforming or transfecting a cell. The invention relates to a host cell comprising a nucleic acid, an expression cassette or a vector encoding a thermostable human IFNγ variant and its use for producing a thermostable recombinant human IFNγ variant according to the invention. The term “host cell” encompasses the daughter cells resulting from the culture or the growth of said cell. In a particular embodiment, the cell is non-human and non-embryonic. The invention also concerns a method for producing a thermostable recombinant human IFNγ variant according to the invention comprising transforming or transfecting a cell with a polynucleotide, an expression cassette or a vector according to the invention; culturing the transfected/transformed cell; and recovering the thermostable human IFNγ variant produced by the cell. In an alternative embodiment, the method for producing a thermostable recombinant human IFNγ variant according to the invention comprises providing a cell comprising a polynucleotide, an expression cassette or a vector according to the invention; culturing the transfected/transformed cell; and recovering the thermostable human IFNγ variant produced by the cell. In particular, the cell may be transformed/transfected in a transient or stable manner by the nucleic acid encoding the variant. Said nucleic acid may be contained in the cell in the form of an episome or in chromosomal form. The methods for producing recombinant proteins are well known to those skilled in the art. Example include the specific methods described in U.S. Pat. No. 5,004,689, EP 446 582, Wang et al. (Sci. Sin. B 24:1076-1084, 1994 and Nature 295, page 503) for production in E. coli, and JAMES et al. (Protein Science (1996), 5:331-340) for production in mammalian cells.

The present invention can further relate to a pharmaceutical composition comprising a nucleic acid encoding a thermostable IFNγ variant according to the invention, an expression cassette, a vector or a host cell according to the invention, its use for preparing a medicament, in particular designed for treating the aforementioned diseases. The invention also relates to a method for treating a patient so requiring it comprising administering such composition in a therapeutically effective amount.

All references cited in this description appear in the list of references of the present application. Other aspects and advantages of the invention will become more apparent in the following examples which of course are given for purposes of illustration and not by way of limitation.

### EXAMPLES

### Selection of Thermostable Human IFNγ Variants

The inventors have developed a method for directly selecting thermostable protein variants called THR and described in French patent application 05 05935.

Said method is based on preparing a fusion protein between the human IFNγ variants and a kanamycin resistance protein variant, having increased thermostability. (This double mutant of kanamycin nucleotidyl transferase is described in Liao, Enzyme Microb. Technol., 1993, 15, 286-92). The library of human IFNγ variants was prepared by the method of Massive Mutagenesis® described in FR2813314, and was transformed at high temperature in Thermus thermophilus strain HB27. Transformed clones from said library were selected at increasing kanamycin concentrations for which production of the (wild-type IFNγ)-KNTase fusion protein no longer allowed cell growth. Theoretically, the clones obtained in said conditions are associated with an increased stability at high temperature, as indicated in French patent application 05 05935 filed on Jun. 10, 2005. The mutations carried on the selected clones were identified and the resulting sequences are listed in Table 1. The different mutants isolated in this first selection were again transformed individually and their level of resistance was compared with the wild-type construct. In this manner, 21 mutants conferring varying degrees of resistance though always greater than wild-type, were confirmed (Table 2).

### Systematic Generation of IFNγ Point Mutants

In addition, starting from the expression clone pORF/IFNγ, a large series of IFNγ point mutations was generated having one and only one amino acid difference relative to the so-called wild-type IFNγ positions (using SEQ No. 6 as reference).

### Functional Analysis of IFNγ Variants

The human IFNγ variants selected by this selection method or systematically generated at all positions of IFNγ were transiently expressed in COS7 animal cells. These proteins were secreted in the culture supernatant. To evaluate the stability and conservation of activity of said variants, the COS7 cell culture supernatants were subjected to thermal denaturation for 10 minutes at 59° C. These proteins (denatured or not) were then made to act on transfected HeLa cells containing the luciferase reporter gene. After 16 hours of stimulation, for each mutant and for each condition, the firefly luciferase signal corresponding to IFNγ activity was measured. The activity induced by the non-denatured protein was then compared with the activity induced by said same denatured protein and the residual activity after thermal denaturation for the protein under study was then calculated (Residual activity=denatured activity/non-denatured activity*100). The basal activity of the non-denatured variant was also compared with that of non-mutated IFNγ.

### Experimental Details:

### Molecular Biology

### Plasmid Constructs

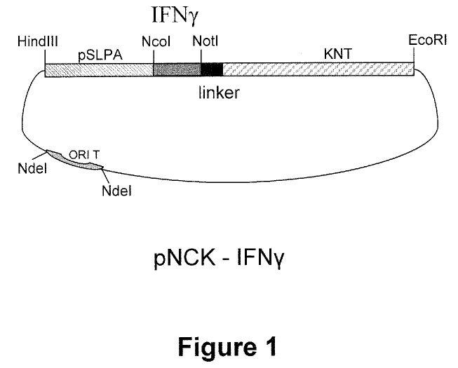

THR system: The vector used contained the E. coli and T. Thermophilus origins of replication, an ampicillin resistance gene for selection of transformants in E. coli, a gene encoding thermostable KNTase under the control of a promoter active in both E. coli and T. thermophilus (the ps1pA promoter). See FIG. 1. The IFNγ nucleotide sequence (coding for the mature form containing 146 amino acids, SEQ ID No. 5) was cloned between the NcoI and NotI sites of the vector on the N-terminal side of KNTase. The IFNγ-KNTase fusion was separated by a linker peptide with the peptide sequence AAAGSSGSI (SEQ ID No. 8) and was coded by the nucleotide sequence GCG-GCC-GCA-GGA-AGC-TCT-GGT-TCC-ATC (SEQ ID No. 7).

Eukaryotic expression system: To express IFNγ in mammalian cells, pORF/IFNγ (Invivogen) was used, in which IFNγ was cloned into an expression cassette containing the hybrid promoter EF-1α-HLTV and the strong SV40 polyadenylation sequence.

### Production of Reference IFNγ Mutants

Several thermostable mutants described in the literature were constructed and used as positive controls. They code for:
the protein IFNγ E30C/S92C: mutant with a disulfide bridge (stability increase of mt +15° C., Waschutza et al., 1996)
the protein IFNγ delta 10 (deletion of the last 10 amino acids at the C-terminal end of the protein, improved activity and stability, mt +7.5° C. and anti-viral activity multiplied four-fold, Slodowski et al., 1991).

Said mutants were produced in pNCK-IFNγ and PORF-IFNγ matrices by the Massive Mutagenesis® method described in FR2813314.

Production in pORF-IFNγ of IFNγ Mutants Selected by the THR Method

Single and multiple mutations corresponding to the mutations identified on the sequences of clones selected by the THR method were introduced into pORF-IFNγ by the Massive Mutagenesis® method described in FR2813314.

Systematic Generation of Single IFNγ Mutations in pORF/IFNγ

Systematic generation of all single IFNγ variants (that is, substitution of the amino acid of the mature wild-type protein by the other 19 amino acids in the genetic code) was carried out in pORF/IFNγ by Massive Mutagenesis® as described in FR2813314.

### Selection of Thermostable Mutants by the THR Method

A library of IFNγ variants cloned into the pNCK vector was produced by Massive Mutagenesis®. Total diversity was introduced at all positions (from 21 to 166). The library was then transformed at high temperature (70° C.) in Thermus thermophilus strain HB27 and selected on 20 or 40 μg/ml kanamycin (conditions where (wild-type IFNγ, did not allow cell growth). The mutations identified by sequencing the clones which grew on selective medium are listed in Table 1. The different mutants isolated in this primary selection were then retransformed individually and their level of resistance was compared with that of the wild-type construct. In this manner 21 mutants, conferring varying degrees of resistance though always greater than wild-type, were confirmed (Table 2).

### Functional Validation of the Stability and Activity of the IFNγ Mutants

All cell culture reagents were from Invitrogen. HeLa cells (human cervix epitheloid carcinoma cells), COS-7 cells (African green monkey SV40 transformed kidney cells) and CHO cells (Chinese Hamster Ovary) were grown in standard culture conditions (37° C. in a humid 5-8% CO₂ atmosphere) respectively in Dulbecco's Modified Eagle's Medium (D-MEM) and Iscove's Modified. Dulbecco's Medium (IMDM). All culture media were prepared according to the supplier's recommendations and contained an L-glutamine analogue (glutamax). Media were supplemented with decomplemented fetal calf serum (FCS) at 10% final concentration for the culture and transfection phases and at a lower percentage concentration for the production phases, depending on the experiment, and with antibiotics (100 units/ml penicillin and 0.1 mg/ml streptomycin for HeLa and COS7 cells and 50 units/ml penicillin and 0.05 mg/ml streptomycin for CHO cells). The pSV-Betagal™ vector (Promega) expressing beta-galactosidase under control of the SV40 early promoter was used to normalize all transfection efficiencies.

### Expression of Human IFNγ Mutants in COS7 Mammalian Cells

For transfection of COS7 cells with the native or mutant pORF/IFNγ constructs, the cells were trypsinized when they reached 90% confluence, then re-seeded at a ratio of 1/4 (in order to obtain approximately 25% confluence after surface adherence). COS7 cells were transfected in 24 well microtiter plates using 30,000-60,000 cells per well when the cells reached 70-80% confluence. Transfection was carried out using approximately 50 ng DNA and Jet PEI (Polyplus transfection) with a Jet PEI/DNA ratio of 5 for 30 minutes at room temperature. After a 24-hour transfection the medium (500

μL IMDM+FCS+antibiotics) was changed. Supernatants containing IFNγ (with a level of expression of about 0.5-1 μg/ml) were recovered at T=24 h post-transfection, aliquoted and stored at −20° C. pending the assay of IFNγ activity.

### Expression of Human IFNγ Mutants in CHO Mammalian Cells

For transfection of CHO cells with the native or mutant pORF/IFNγ constructs, the cells were trypsinized when they reached 80% confluence, then re-seeded at a ratio of 1/3 (in order to obtain approximately 33% confluence after surface adherence). CHO cells were transfected when they reached 70% confluence, in a T75 flask with 4.10⁶ cells per flask. Transfection was carried out using approximately 8 μg DNA and Jet PEI (Polyplus transfection) with a Jet PEI/DNA ratio of 5 for 30 minutes at room temperature. After a 24-hour transfection the medium (10 ml IMDM+2.5% FCS+antibiotics) was removed, cells were washed briefly with 1×PBS, and the medium was replaced with IMDM+antibiotics and no additional FSC. Supernatants containing IFNγ (with a level of expression of about 0.5-1 μg/ml) were recovered at T=48 h post-transfection, aliquoted and stored at −20° C. pending the assay of IFNγ activity.

### Quantification of Wild-Type and Variant Human IFNγ

The reference ELISA kit for determining the total quantity of IFNγ was from Clinisciences (# 88-7316-86). It was verified that the antibodies used in this ELISA kit recognized our variants and the non-mutant native molecule in the same way by quantifying said same proteins with another commercial ELISA kit (Biosource # KHC4021).

### Primary Activity Test

It has previously been shown that IFNγ specifically activates IFNγ receptors on HeLa cells. IFNγ-mediated stimulation of the Jak/Stat1 pathway in HeLa cells results, in particular, in the transcriptional activation of genes under the control of promoters containing GAS (Gamma Activated Site) sequences. It is thus possible to measure and compare the activities of IFNγ variants by transfecting HeLa cells with a reporter gene system in which luciferase (firefly luciferase) is cloned downstream of a promoter containing several GAS sites (plasmid pGAS/Luciferase from Stratagene).

Transient Transfection of HeLa Cells with pGAS/Luciferase

HeLa cells were trypsinized when they reached 90% confluence and then re-seeded at a ratio of 1/3. Transfections of HeLa cells at 50-80% confluence were carried out in 96-well microtiter plates according to the supplier's protocol: 20,000 cells per well were transfected with approximately 150 ng of pGAS/Luciferase DNA and Jet PEI at a Jet PEI/DNA ratio of 5, then vortexed for 30 seconds and left at room temperature for 30 minutes. Twenty microliters of the DNA/Jet PEI mixture were then aliquoted into each well of the plate and cells so transfected were cultured for 24 hours at 37° C. in a 5% CO₂ atmosphere.

### Primary Screening of Thermostable Human IFNγ Variants

### Measurement of Total Basal Activity of IFNγ Variants (Non-Denatured Protein)/(Wild-Type Protein):

COS7 cell supernatants containing IFNγ were diluted 1 to 100 and 10 μl of these dilutions were added to HeLa cells transfected with pGAS/Luciferase. The plates were incubated for 16 hours at 37° C. in a 5% CO₂ atmosphere to allow cytoplasmic expression of firefly luciferase and cell pellets were then recovered and frozen. Fifty microliters of Glo lysis Buffer™ (Promega) were added to lyse the cells (10 minutes with shaking at room temperature so as to release the luciferase produced in response to specific stimulation by IFNγ). Activity was measured by adding Bright Glo™ reagent (Promega) and the quantity of accumulated luciferase was counted on a luminometer (FLX 800, Bio-Tek Instrument). Crude IFNγ activity is expressed in RLU (relative luciferase units). Total activity (relative to the wild-type protein) of each variant (non-denatured) was calculated as a mean of data from five different experiments (duplicates at a minimum) and on culture supernatants from a minimum of two independent transfections. The error bars were calculated by the standard error of the mean (s.e.m). One way to present the total activity data is to report the basal activity of each variant as a percentage of the basal activity of the non-mutant IFNγ expressed in the same conditions for each transfection.

Measurement of Residual Activity of the Variants after Thermal Denaturation Using the Luciferase Reporter Gene:

As described earlier for the primary activity test using the reporter gene, the quantity of luciferase was measured after stimulating the cells with 10 μl of a 1/100 dilution of COS7 cell supernatant containing IFNγ which, according to the case, was subjected to thermal denaturation treatment at 59° C. for 10 minutes, or left untreated. One way to express the fraction of activity of each variant remaining after thermal denaturation is to calculate the residual activity conserved by each variant defined as the percentage of the basal activity of the same variant before denaturation. Residual activity relative to total activity before denaturation was calculated as a mean of the data from five different experiments (duplicates at a minimum) and on culture supernatants from a minimum of two independent transfections. The error bars were calculated using the formula for the standard error of the mean (s.e.m).

Measurement of an Improvement Index for the IFNγ Variants (Improvement of Thermostability and/or Activity):

Once it was verified that the IFNγ variant in question showed both improved thermostability and at least partially conserved the intrinsic activity of the protein, one could calculate the product of the residual activity of the variant after pretreatment, multiplied by its activity (without pretreatment). This index gives an idea of the gain in thermostability and the relative loss of activity, and therefore an idea of the expected in vivo effect of the protein. The following values were obtained with the reference proteins: an index of 37 for non-mutant IFNγ and approximately 76 for the delta 10 mutant of the literature.

Upon completion of the primary screening, a series of human IFNγ variants was identified having improved thermostability and/or activity as compared with the non-mutated human IFNγ, as shown in FIGS. 2 to 6.

### Secondary Screening of Thermostable Human IFNγ Variants—Measurement of In Vitro Half-Life

With a view to comparing the improvement in the intrinsic stability of the most thermostable IFNγ variants identified in the primary screening, a secondary screening was carried out to follow the variation in the activity of the IFNγ variants as a function of thermal denaturation time at 59° C. In this manner a half-life at 59° C. was determined, herein denoted “in vitro half-life”, which corresponds to the denaturation time required to reduce the initial IFNγ activity by 50% at 59° C. This half-life was determined by controlling in particular all the following parameters: a same IFNγ starting concentration of 1000 pg/ml, an equivalent serum FCS concentration in the final sample to be denatured and adjusted to 0.15%, and a denaturation temperature of 59° C. for 30 minutes with samples taken every 10 minutes.

IFNγ concentrations in the CHO cell supernatants were determined by ELISA. These different groups of IFNγ variants were then diluted to 1000 pg/ml in IMDM medium +0.15% FCS. The dilutions were aliquoted so as to undergo thermal denaturation pretreatment at 59° C. for 0, 10, 20 and 30 minutes. Ten microliters of the pretreated dilutions were added to 100 μl of medium from HeLa cells transfected with pGAS/Luciferase as described earlier. After 16 hours at 37° C. in a 5% CO₂ atmosphere, cell pellets were lysed and luciferase quantified as described earlier.

One way to present the data is to report the crude activity corresponding to specific stimulation of the transduction pathway by the IFNγ variant, said crude activity being expressed in RLU (relative luciferase units). Another way to present the fraction of activity conserved after thermal denaturation (also called residual activity) of each variant is to calculate, for each denaturation time, the percentage of residual activity conserved relative to the basal activity of the same variant before denaturation. These calculations were made after subtracting the signal from non-transfected cells. The half-lives of each variant determined in this way were then compared with that of wild-type IFNγ. Half-lives (T1/2) are expressed in minutes and calculated with the aid of the following formula:

T1/2=ln(2)/(k inactivation)

where k inactivation is the velocity constant of the inactivation phase.

Said constant is calculated on the mean of the instantaneous inactivation rates of each time point according to the following formula:

Ln A(t)=ln b−t*(k inactivation)

A (t) is IFNγ activity at time t and b is a constant.

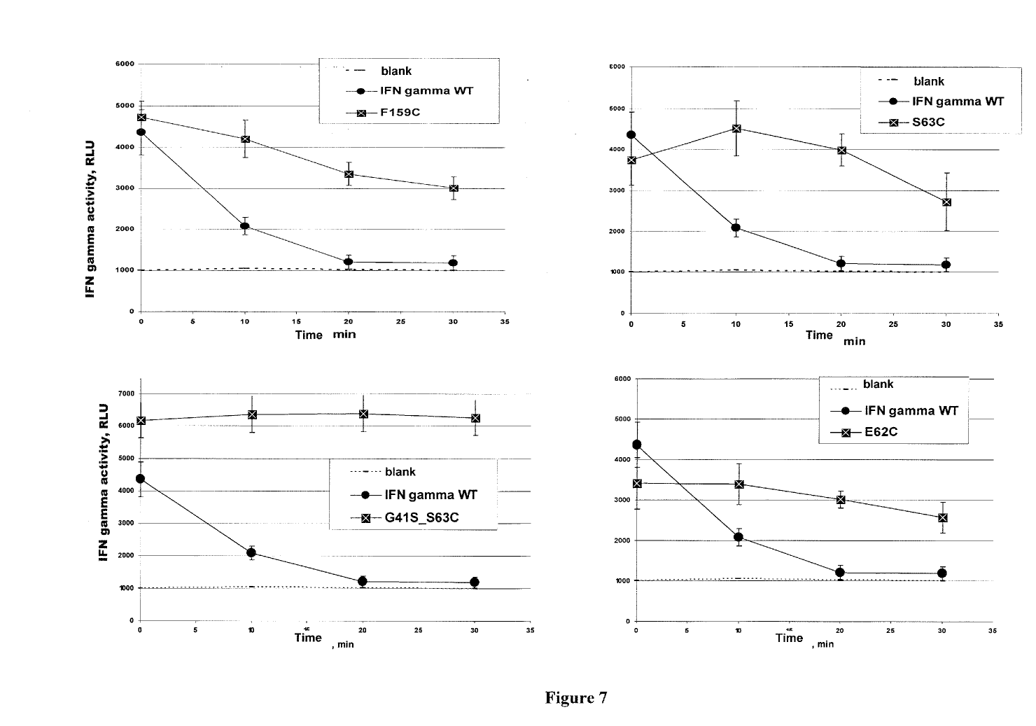

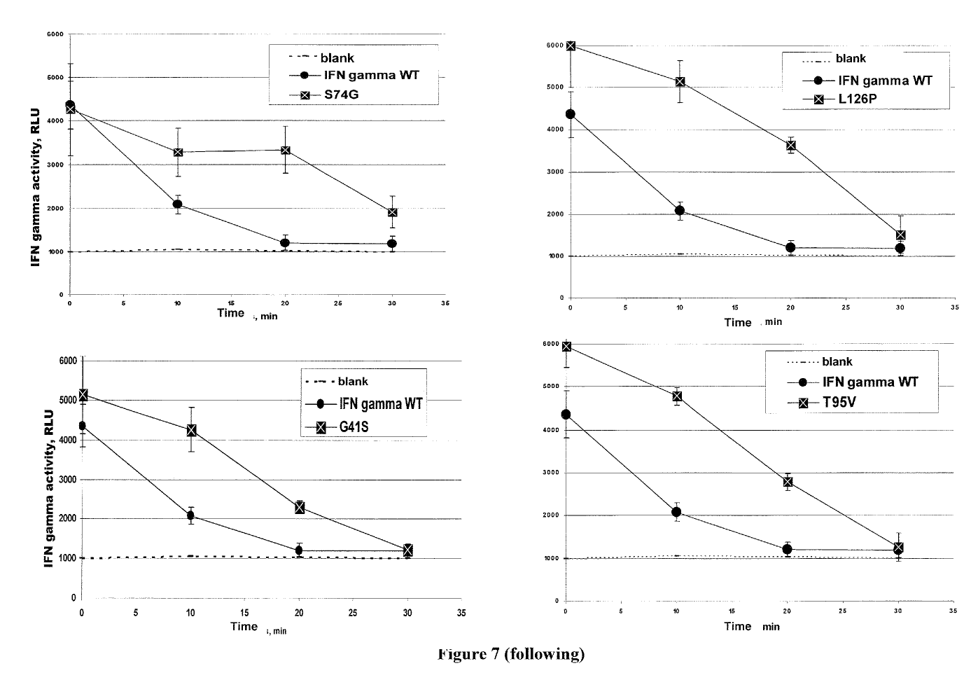

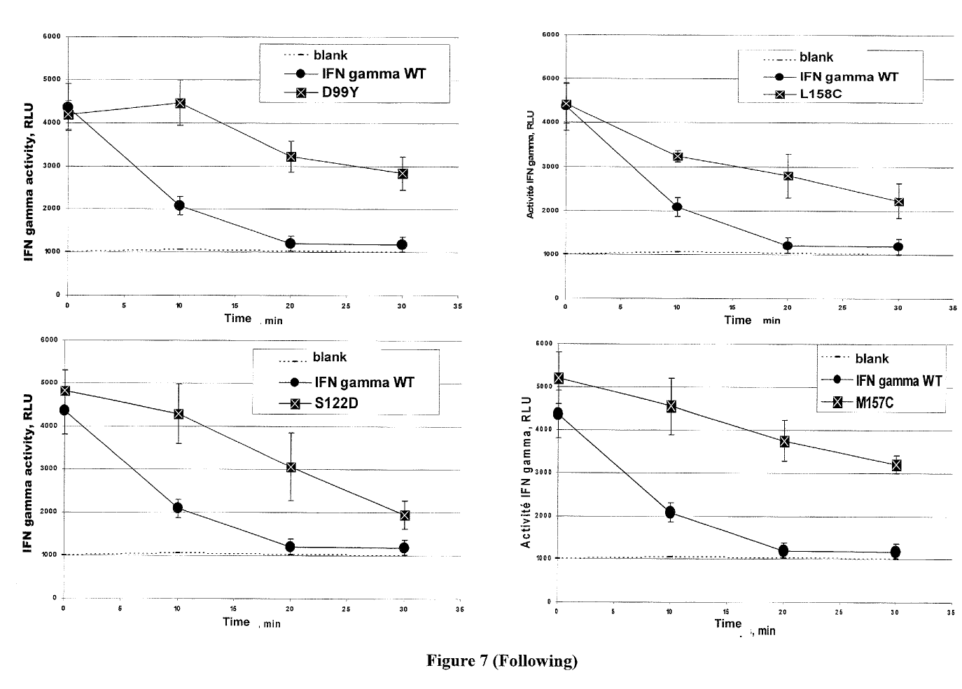

So, to summarize, the “in vitro” half-lives calculated herein, presented in FIG. 7 and summarized in Table 3, correspond to the time during which half of the maximum activity of each IFNγ variant was conserved.

Another parameter presented in Table 3 is the ratio of improvement in the half-life of each variant relative to the non-mutant wild-type molecule produced in the same conditions. The higher the value of this parameter, the greater the improvement in the “in vitro” half-life of the variant compared with that of non-mutant human IFNγ.

Thus, upon completion of this secondary screening, 16 IFNγ mutants were isolated showing a marked improvement in their in vitro half-life, by a factor of 200 to 1.4-fold, as summarized in FIG. 7 and Table 3.

### Measurement of the Pharmacokinetic Parameters of Thermostable Human IFNγ Variants in Mice

These descriptive pharmacokinetic studies of thermostable IFNγ variants aimed to determine the biological half-life or terminal elimination half-life (also called “in vivo half-life”), as well as the areas under the curve for the different routes of administration (intravenous (i.v.) or subcutaneous (s.c.) injection). These data were then compared with those obtained for wild-type IFNγ produced in mammalian cells and in the case of standard recombinant IFNγ produced in E. coli displaying the characteristics of the commercial molecule, in other words, a pseudo “Actimmune”.

The measurement of biological half-life can be carried out in a number of ways:

One method described by Rutenfranz et al. (J. Interferon Res. 1990, vol. 10, p. 337-341) used intravenous and intramuscular injection in 8-week-old C57BL/6 mice.

The half-life was measured by an antiviral activity test directly on murine serum (Hep2 cells infected by vesicular stomatitis virus or VVS). In this example, ELISA was used as an alternative to detect IFNγ levels in murine serum.

Another method described by Croos and Roberts (J. Pharm., 1993, vol 45, p. 606609) used radiolabelled IFNγ and followed its absorption in tissues and its levels in serum of female Sprague-Dawley rats after subcutaneous injection. Blood and tissue samples were analyzed for the amount of radiolabelled IFNγ they contained.

The use of an ELISA method to determine the pharmacokinetic parameters of IFN has been described for IFN alpha after subcutaneous administration by Rostaing et al. (1998), J. Am. Soc. Nephro1.9 (12): 2344-48 and after intramuscular administration by Merimsky et al. (1991), Cancer Chemother. Pharmacol. 27 (5).]

Wild-type and mutant IFNγ were expressed in COS and CHO cells. The culture supernatants were centrifuged a second time at 4000 rpm for 10 minutes, then filtered on a Millipore PES 0.22 μm filter. The filtered supernatants were concentrated by centrifugation on a Vivaspin filtration unit with a 5000 dalton cutoff (Sartorius). IFNγ concentrations were then determined on the samples by using the previously adapted dilutions.

Wild-type and mutant IFNγ were administered in two ways:
by injection of 100 μl of a 10 μg/ml IFNγ solution into the caudal veins of C57BL/6J mice.
or else by subcutaneous injection of 100 μl of a 6.7 μg/ml IFNγ solution into the stomach of C57BL/6J mice.

These experiments used 8-week-old C57BL/6J weighing between 20 and 30 grams. The animals were acclimated for one week in a room at a constant temperature of 24.1° C., constant 55% humidity and a 12 hour day/night cycle.

Blood samples were collected at different times after administration of the molecule of interest. Retro-orbital samples were taken at 3, 6, 24, 48, 72, 96, 120, 144, 168 and 192 hours and postmortem cardiac samples at the final time point of 216 hours.

Serum was prepared by allowing the blood to clot for 20 minutes at room temperature and recovering the fraction corresponding to the supernatant of a centrifugation at 5000 g for 20 minutes at 20° C. Serum was then isolated and stored at −80° C. until measurement of IFNγ activity by the ELISA assay described earlier.

Plasma IFNγ concentrations were then measured over time by following the quantity of IFNγ detected by ELISA in each mouse serum. For each retro-orbital sample, IFNγ was quantified as the mean of at least three different sera from three mice. These different pharmacokinetic experiments were carried out for each variant at least twice using IFNγ from different transfections. IFNγ concentrations in serum over time were then reported.

The parameters [T_{1/2 i.v}, AUC_{i.v},] [T_{1/2sc} et AUC_{sc}], were calculated with the aid of Kinetica software (Vs 4.4.1 Thermo Electron Inc.) respectively using an “intravenous bolus non-compartmental” and an “extravascular bolus” pharmacokinetic analytical model.

Another parameter presented in Tables 4 and 5 is the ratio of improvement in the half-life or area under the curve for each variant relative to the respective parameters of the non-mutant wild-type molecule produced in the same conditions, and relative to the respective parameters of bacterial recombinant human IFNγ when such data were available. The higher these ratios, the greater the improvement in the variant as compared with that of non-mutant human IFNγ.

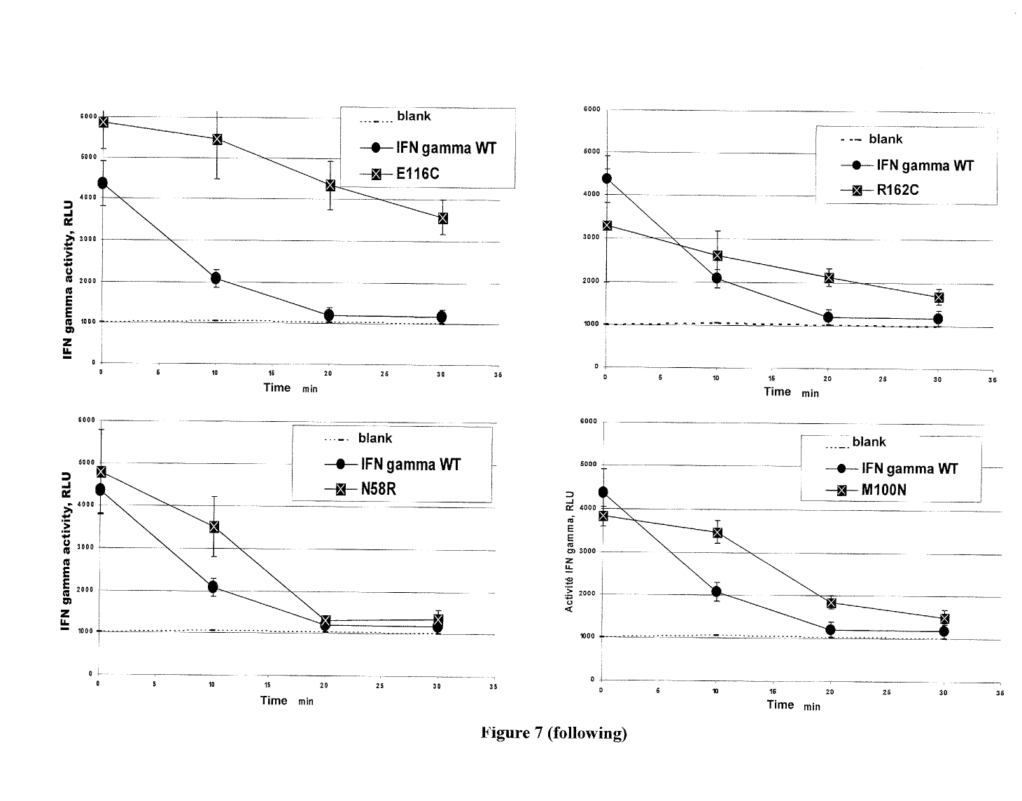

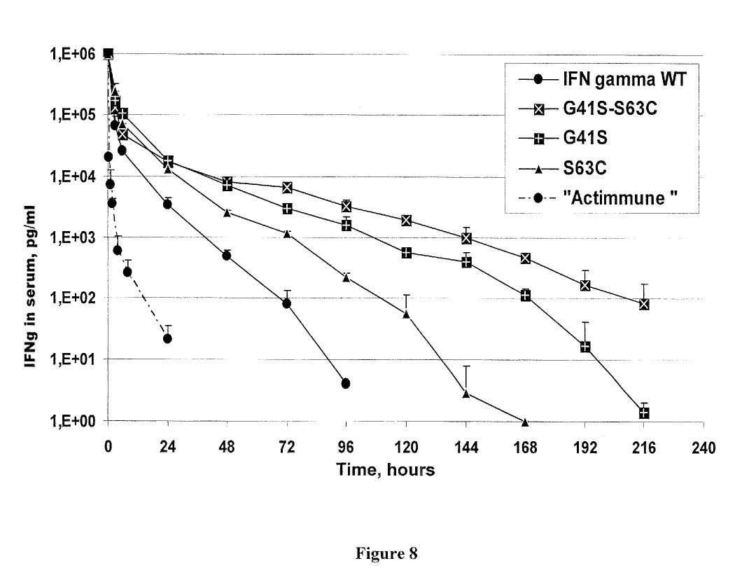

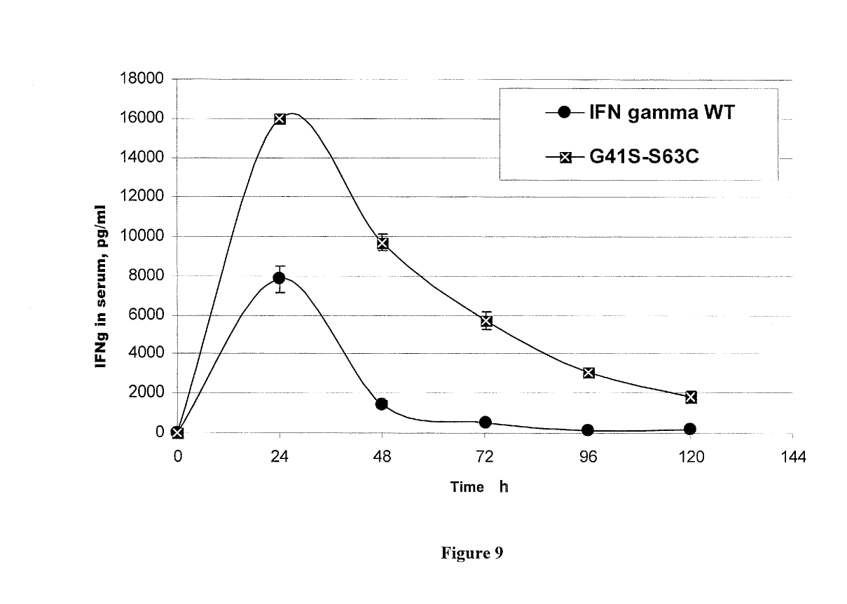

These experimental results and their analysis are presented in FIG. 8 and Table 4 in the case of intravenous injections and in FIG. 9 and Table 5 for subcutaneous injections.

It can be seen that the mutations S63C, G41S and the combination thereof resulted in a marked improvement in the in vivo half-life of these variants compared with that of recombinant human IFNγ produced in CHO cells and with that of recombinant human IFNγ produced in bacteria. After subcutaneous administration, the double mutant again showed a marked improvement in these pharmacokinetic parameters compared with those of recombinant human IFNγ produced in CHO cells.

TABLE 1

| Name | mutations position (aa) | | | | |
|---|---|---|---|---|---|
| 1 | 147: GCT→GAA (Ala→Glu) | 162: CGA→GAC (Arg→Asp) | | | |
| 2 | 162: CGA→GAA (Arg→Glu) | | | | |
| 4 | 163: AGA→ACC (Arg→Thr) | | | | |
| 5 | 21: TGT→GGC (Cys→Gly) | 159: TTT→TGC (Phe→Cys) | | | |
| 8 | 100: ATG→AAC (Met→Asn) | 119: ACT→TAC (Thr→Tyr) | | | |
| 9 | 24: CAG→GCG (Gln→Ala) | | | | |
| 10 | 22: TAC→GAC (Tyr→Asp) | 122: TCG→CAC (Ser→His) | | | |
| 12 | 25: GAC→GTC (Asp→Val) | | | | |
| 13 | 76: TAC→GAC (Tyr→Asp) | 131: AAA→ATC (Lys→Ile) | | | |
| 14 | 22: TAC→ACG (Tyr→Thr) | 109: AAA→TGC (Lys→Cys) | 119: ACT→CCC (Thr→Pro) | 147: GCT→TTC (Ala→Phe) | 163: AGA→CTC (Arg→Leu) |
| 15 | 50: ACT→TAC (Thr→Tyr) | 121: TAT→ACC (Tyr→Thr) | 140: ATG→CCG (Met→Pro) | | |
| 17 | 28: GTA→TGC (Val→Cys) | | | | |
| 18 | 22: TAC→CTC (Tyr→Leu) | 68: ATG→TTC (Met→Phe) | 86: GAC→CAG (Asp→Gln) | 100: ATG→TGG (Met→Trp) | 119: ACT→AGG (Thr→Arg) |
| 19 | 21: TGT→GAG (Cys→Glu) | 45: GTA→GGT (Val→Gly) | 65: AGA→ATC (Arg→Ile) | 71: CAA→TGC (Gln→Cys) | 89: ATC→TTC (Ile→Phe) |
| 20 | 68: ATG→CTG (Met→Leu) | | | | |
| 21 | 26: CCA→GAC (Pro→Asp) | 122: TCG→CCG (Ser→Pro) | | | |
| 22 | 165: TCC→GTG (Ser→Val) | | | | |
| 23 | 120: AAT→ATG (Asn→Met) | | | | |
| 26 | 21: TGT→CAG (Cys→CAG) | 90: CAA→CAT (Gln→Asn) | | | |
| 28 | 22: TAC→TCC (Tyr→Ser) | 158: CTG→TGG (Leu→Trp) | 163: AGA→GGA (Arg→Gly) | | |
| 31 | 124: ACT→CGG (Thr→Arg) | | | | |
| 32 | 164: GCA→GAG (A1a→Glu) | | | | |
| 34 | 126: TTG→CAT (Leu→His) | 157: ATG→TGG (Met→Trp) | | | |
| 35 | 22: TAC→TCC (Tyr→Ser) | 158: CTG→TGG (Leu→Trp) | | | |
| 36 | 162: CGA→CAA (Arg→Gln) | 164: GCA→GAG (Ala→Glu) | | | |
| 6 | 21: TGT→TGG (Cys→Trp) | | | | |
| 7 | 21: TGT→GGC (Cys→Gly) | | | | |
| 11 | 25: GAC→CAC (Asp→His) | | | | |
| 24 | 22: TAC→TGG (Tyr→Trp) | | | | |
| 25 | 22: TAC→TTC (Tyr→Phe) | | | | |
| 27 | 21: TGT→GAG (Cys→Glu) | | | | |
| 29 | 21: TGT→GCC (Cys→Ala) | | | | |
| 30 | 59: TGG→TT (Trp→Phe) | | | | |
| 33 | 51: CTT→TTC (Leu→Phe) | | | | |
| 37 | 98: GAA→AAA (Glu→Lys) | | | | |
| 38 | 63: AGT→AGG (Ser→Arg) | | | | |
| 39 | 28: GTA→TGC (Val→Cys) | | | | |

TABLE 2

| Clone N° | Mutated positions | | | | |
|---|---|---|---|---|---|
| 1 | 147: GCT→GAA (Ala→Glu) | 162: CGA→GAC (Arg→Asp) | | | |
| 2 | 162: CGA→GAA (Arg→Glu) | | | | |
| 4 | 163: AGA→ACC (Arg→Thr) | | | | |
| 5 | 21: TGT→GGC (Cys→Gly) | 159: TTT→TGC (Phe→Cys) | | | |
| 6 | 21: TGT→TGG (Cys→Trp) | | | | |
| 8 | 100: ATG→AAC (Met→Asn) | 119: ACT→TAC (Thr→Tyr) | | | |
| 9 | 24: CAG→GCG (Gln→Ala) | | | | |
| 12 | 25: GAC→GTC (Asp→Val) | | | | |
| 13 | 76: TAC→GAC (Tyr→Asp) | 131: AAA→ATC (Lys→Ile) | | | |
| 15 | 50: ACT→TAC (Thr→Tyr) | 121: TAT→ACC (Tyr→Thr) | 140: ATG→CCG (Met→Pro) | | |
| 21 | 26: CCA→GAC (Pro→Asp) | 122: TCG→CCG (Ser→Pro) | | | |
| 37 | 98: GAA→AAA (Glu→Lys) | | | | |
| 39 | 28: GTA→TGC (Val→Cys) | | | | |
| 22 | 165: TCC→GTG (Ser→Val) | | | | |
| 28 | 22: TAC→TCC (Tyr→Ser) | 158: CTG→TGG (Leu→Trp) | 163: AGA→GGA (Arg→Gly) | | |
| 10 | 22: TAC→GAC (Tyr→Asp) | 122: TCG→CAC (Ser→His) | | | |
| 34 | 126: TTG→CAT (Leu→His) | 157: ATG→TGG (Met→Trp) | | | |
| 36 | 162: CGA→CAA (Arg→Gln) | 164: GCA→GAG (Ala→Glu) | | | |
| 14 | 22: TAC→ACG (Tyr→Thr) | 109: AAA→TGC (Lys→Cys) | 119: ACT→CCC (Thr→Pro) | 147: GCT→TTC (Ala→Phe) | 163: AGA→CTC (Arg→Leu) |
| 30 | 59: TGG→TT (Trp→Phe) | | | | |
| 38 | 60: AGT→AGG (Ser→Arg) | | | | |

TABLE 3

| | | Improvement Ratio of |
|---|---|---|
| | T½, min In vitro | variant half-life/WT? |
| | half-life at 59° C. | half-life produced in CHO |
| Non-mutated IFN gamma | 5 | 1 |
| (produced in CHO) | | |
| G41S_S63C | 981 | 200.6 |
| S63C | 50 | 10.3 |
| E62C | 53 | 10.9 |
| F159C | 33 | 6.7 |
| D99Y | 32 | 6.6 |
| E116C | 31 | 6.4 |
| M157C | 31 | 6.4 |
| L158C | 21 | 4.2 |
| S74G | 18 | 3.6 |
| R162C | 17 | 3.4 |
| S122D | 14 | 3.0 |
| M100N | 11 | 2.2 |
| L126P | 9 | 1.9 |
| N58R | 7 | 1.4 |
| T95V | 7 | 1.4 |
| G41S | 7 | 1.4 |

TABLE 4

| | | Improvement Ratios | |
|---|---|---|---|
| | | T½ i.v mutant/ | T½ i.v mutant/ |
| | | T½ i.v | T½ i.v |
| | T½ i.v, h | << Actimmune >> | WT CHO |
| Bacterian | 4.5 | 1 | — |
| recombinant | | | |
| IFN gamma | | | |
| IFN gamma | 7.4 | 1.7 | 1 |
| WT, CHO | | | |
| G41S-S63C | 25.3 | 5.6 | 3.4 |
| G41S | 15 | 3.3 | 2 |
| S63C | 13.4 | 3 | 1.8 |
| L158C | 8.7 | 1.9 | 1.2 |
| F159C | 8.0 | 1.8 | 1.1 |
| S122D | 7.8 | 1.7 | 1.1 |
| E116C | 8.3 | 1.8 | 1.1 |
| E62C | 7.5 | 1.7 | 1.0 |
| D99Y | 7.3 | 1.6 | 1.0 |
| M157C | 7.4 | 1.6 | 1.0 |
| L126P | 7.6 | 1.7 | 1.0 |
| T95V | 7.7 | 1.7 | 1.0 |
| R162C | 7.0 | 1.6 | 0.9 |

TABLE 5

| | | Ratio AUC | | Ratio T½ |
|---|---|---|---|---|
| Dose | AUC tot. | tot s.c/ | | s.c/ |
| 670 ng | s.c, | WT CHO | T½. s.c, h | WT CHO |
| IFNγ WT CHO | 222140 | — | 12.3 | — |
| G41S-S63C | 914660 | 4.1 | 30.3 | 2.5 |

1-32. (canceled)

33. A pharmaceutical composition comprising a thermostable variant of human interferon gamma (IFNγ) or a functional fragment thereof comprising at least a first amino acid substitution selected in the group consisting of S63C, E62C, F159C, D99Y, E116C, L158C, S74G, R162C, S122D, L126P, N58R, and T95V, the indicated amino acid positions corresponding to those of SEQ ID No. 2, and wherein the variant not containing a non-peptide moiety attached to the residue(s) introduced by the first substitution(s).

34. The pharmaceutical composition according to claim 33, wherein the variant differs from a polypeptide having the sequence of SEQ ID No. 2, 4 or 6 by at least 1, 2, 3, 4, 5, 6, 7, 8, 9 or 10 amino acid residue(s).

35. The pharmaceutical composition according to claim 33, wherein the variant has a single amino acid substitution.

36. The pharmaceutical composition according to claim 33, wherein the variant additionally comprises at least one other amino acid substitution selected from the group consisting of M157C, G41S and M100N, wherein the indicated amino acid positions corresponding to those of SEQ ID No. 2.

37. The pharmaceutical composition according to claim 36, wherein the variant comprises or has a combination of two amino acid substitutions selected from the group consisting of S63C, E62C, F159C, D99Y, E116C, L158C, S74G, R162C, S122D, M100N, L126P, N58R, T95V, M157C and G41S, the indicated amino acid positions corresponding to those of SEQ ID No. 2.

38. The pharmaceutical composition according to claim 37, wherein the variant comprises or has a combination of amino acid substitutions selected from the group consisting of S63C+E62C, S63C+F159C, S63C+D99Y, S63C+E116C, S63C+L158C, 563C+S740, S63C+R162C, S63C+S122D, S63C+M100N, S63C+L126P, S63C+N58R, S63C+T95V, S63C+M157C, S63C+G41S, E62C+F159C, E62C+D99Y, E62C+E116C, E62C+L158C, E62C+S74G, E62C+R162C, E62C+S122D, E62C+M100N, E62C+L126P, E62C+N58R, E62C+T95V, E62C+M157C, E62C+G41S, F159C+D99Y, F159C+E116C, F159C+L158C, F159C+S74G, F159C+R162C, F159C+S122D, F159C+M100N, F159C+L126P, F159C+N58R, F159C+T95V, F159C+M157C, F159C+G41S, D99Y+E116C, D99Y+L158C, D99Y+S74G, D99Y+R162C, D99Y+S122D, D99Y+M100N, D99Y+L126P, D99Y+N58R, D99Y+T95V, D99Y+M157C, D99Y+G41S, E116C+L158C, E116C+S74G, E116C+R162C, E116C+S122D, E116C+M100N, E116C+L126P, E116C+N58R, E116C+T95V, E116C+M157C, E116C+G41S, L158C+S74G, L158C+R162C, L158C+S122D, L158C+M100N, L158C+L126P, L158C+N58R, L158C+T95V, L158C+M157C, L158C+G41S, 574G+R162C, S74G+S122D, S74G+M100N, S74G+L126P, S74G+N58R, S74G+T95V, S74G+M157C, S74G+G41S, R162C+S122D, R162C+M100N, R162C+L126P, R162C+N58R, R162C+T95V, R162C+M157C, R162C+G41S, S122D+L126P, S122D+N58R, S122D+T95V, S122D+M157C, S122D+M100N, S122D+G41S, L126P+N58R, L126P+T95V, L126P+M157C, L126P+M100N, L126P+G41S, N58R+T95V, N58R+M157C, N58R+M100N, N58R+41S, T95V+M157C, T95V+M100N, T95V+G41S, M157C+M100N and M157C+G41S, the indicated amino acid positions corresponding to those of SEQ ID No. 2.

39. The pharmaceutical composition according to claim 38, wherein the variant comprises or has a combination of substitutions selected from the group consisting of S63C+E62C, S63C+F159C, S63C+D99Y, S63C+E116C, S63C+L158C, S63C+S74G, S63C+R162C, S63C+S122D, S63C+M100N, S63C+L126P, S63C+N58R, S63C+T95V, S63C+M157C, and S63C+G41S, the indicated amino acid positions corresponding to those of SEQ ID No. 2.

40. The pharmaceutical composition according to claim 39, wherein the variant comprises or has the combination of amino acid substitution S63C+G41S, the indicated amino acid positions corresponding to those of SEQ ID No. 2.

41. The pharmaceutical composition according to claim 33, wherein the variant does not have a deletion of 1 to 11 residues at the C-terminal end.

42. The pharmaceutical composition according to claim 33, wherein the variant has a deletion of 1 to 11 residues at the C-terminal end.

43. The pharmaceutical composition according to claim 33, wherein the variant does not contain any non-peptide moiety selected from the group consisting of a polymer molecule, a lipophilic molecule, and an organic derivatizing agent.

44. The pharmaceutical composition according to claim 33, wherein the variant contains a non-peptide moiety selected from the group consisting of a polymer molecule, a lipophilic molecule, and an organic derivatizing agent.

45. The pharmaceutical composition according to claim 44, wherein the non-peptide moiety is a polymer molecule.

46. The pharmaceutical composition according to claim 33, wherein the variant is glycosylated.

47. The pharmaceutical composition according to claim 33, wherein the variant is not glycosylated.

48. The pharmaceutical composition according to claim 33, additionally comprising at least one other active agent.

49. The pharmaceutical composition according to claim 48, wherein the at least one other active agent is selected from the group consisting of an antibody, an antitumoral or chemotherapeutic agent, a glucocorticoid, an antihistamine, an adrenocortical hormone, an anti-allergic agent, a vaccine, a bronchodilator, a steroid, a beta-adrenergic agent, an immunomodulating agent, interferon alpha or beta, interleukin 1 or 2, TNF (tumor necrosis factor), hydroxyurea, an alkylating agent, a folic acid antagonist, a nucleic acid antimetabolite, a spindle poison, an antibiotic, a nucleotide analogue, a retinoid, a lipoxygenase and cyclooxygenase inhibitor, fumaric acid and its salts, an analgesic, a spasmolytic, and a calcium antagonist.

50. The pharmaceutical composition according to claim 49, wherein the at least one other active agent is an interferon alpha or beta.

51. The pharmaceutical composition according to claim 33 formulated for oral, parenteral, sublingual, topical, local, intratracheal, intranasal, transdennal, rectal, intraocular or intra-auricular administration.

52. A product comprising a pharmaceutical composition according to claim 33 and another active agent said product being formulated for simultaneous, sequential or separate use.

53. The product according to claim 52, wherein the other active agent is selected from the group consisting of an antibody, an antitumoral or chemotherapeutic agent, a glucocorticoid, an antihistamine, an adrenocortical hormone, an anti-allergic agent, a vaccine, a bronchodilator, a steroid, a beta-adrenergic agent, an immunomodulating agent, interferon alpha or beta, interleukin 1 or 2, TNF (tumor necrosis factor), hydroxyurea, an alkylating agent, a folic acid antagonist, a nucleic acid antimetabolite, a spindle poison, an antibiotic, a nucleotide analogue, a retinoid, a lipoxygenase and cyclooxygenase inhibitor, fumaric acid and its salts, an analgesic, a spasmolytic, and a calcium antagonist.

54. The product according to claim 53, wherein the other active agent is interferon alpha or beta.

55. The product according to claim 53, wherein the two active agents are administered by the same route of administration or by two different routes of administration.

56. A method for treating a pathology selected in the group consisting of asthma, chronic familial granulomatous disease, idiopathic pulmonary fibrosis, an atypical mycobacterial infection, kidney cancer, osteopetrosis, systemic scleroderma, chronic hepatitis B or C, septic shock, allergic dermatitis, and rheumatoid arthritis in a subject, comprising administering a composition of claim 33 or a product of claim 53 in a therapeutically effective amount.

57. A nucleic acid encoding a thermostable IFNγ variant as described in claim 33.

58. An expression cassette, a vector or a host cell comprising a nucleic acid according to claim 57.

59. A method for producing a thermostable recombinant human IFNγ variant according to claim 33 comprising:
a) providing a host cell according to claim 58;
b) culturing the host cell; and
c) recovering the thermostable human IFNγ variant produced by the host cell.


SEQUENCE LISTING (8 sequences)

SEQ ID NO: 1 - DNA, 501 bp, Homo sapiens
  CDS (1)..(501)
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 2 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 3 - DNA, 429 bp, Homo sapiens
  CDS (1)..(429)
caggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccag

SEQ ID NO: 4 - protein, 143 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 5 - DNA, 438 bp, Homo sapiens
  CDS (1)..(438)
tgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccag

SEQ ID NO: 6 - protein, 146 aa, Homo sapiens
CYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 7 - DNA, 27 bp, artificial sequence
  linker
gcggccgcaggaagctctggttccatc

SEQ ID NO: 8 - protein, 9 aa, artificial sequence
  Synthetic Construct
AAAGSSGSI